SEQUENCE LISTING (28 sequences)

SEQ ID NO: 1 - DNA, 3822 bp, Severe acute respiratory syndrome-related coronavirus
  source 1..3822: mol_type = other DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, Severe acute respiratory syndrome-related coronavirus
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 1393 bp, Human immunodeficiency virus 1
  source 1..1393: mol_type = other DNA
gttcctgtgtggaaagatgcagagaccaccttattttgtgcatcagatgccaaagcacatgagacagaagtgcacaatgtctgggccacacatgcctgtgtacccacagaccccaacccacaagaaatacaactgaaaaatgtaacagagaattttaacatgtggaaaaataacatggtagagcagatgcaggaggatgtaatcagtttatgggatcaaagtctaaagccatgtgtaaagttaactcctctctgcgttactttaaattgtaccgatgctactttgaccaatagcacttacataaccaatgtctctaagataataggagatataacagaggaagtaagaaactgttcttttaatatgaccacagaactaagagataagaagcagaaggtccatgcactttttttataagcttgatatagtagaaattgaaaagaataggaatgagtataggttaataaattgtaatacttcggtcattaagcaggcttgtccaaagatatcctttgatccaattcctatacattattgtactccagctggttatgcgattttaaagtgtaatgataagaatttcaatgggacagggccatgtaaaaatgtcagctcagtacaatgcacacatggaattaagccagtggtatcaactcaattgctgttaaatggcagtctagcagaagaagagataataatcagatctgaaaatctcacaaacaatgccaaaaccataatagtgcaccttaataaatctgtagaaatcaattgtaccagacccttcaacaatacaagaacaagtataactataggaccaggacaaatgttctatagaacaggagagataataggagatataacaaaagcatattgtgagattaatggaacaaaatggaatgaaactttaaaacaggtagctgaaaaactaaaagagcactttaataataagacaatagtctttcaaccaccctcaggaggagatctagaaattacaatgcatcattttaattgtagaggggaatttttctattgcaatacaacacgactgtttaatcatacttacatggaaaataaaaccatgggggggtgtaatgacactatcatacttccatgcaagataaagaaaattataaatatgtggcagggagtaggacaagcaatgtatgctcctcccatcaggggaagcattaattgtgtatcaaatattacaggaatactattgataagagatggtggtgataataatgcgactaacgagaccttcagacctggaggaggaaatataaaggacaattggagaagtgaattatataaatataaagtagtacaaattgaaccactaggaatagcacccaccagggcaaagggaagagtggtggagagagaaagaaga

SEQ ID NO: 4 - DNA, 372 bp, Human immunodeficiency virus 1
  source 1..372: mol_type = other DNA
aacaacatatgttgcaactcacagtctggggcataaagcagctccaggcaagagtcctggctgtggaaagatacctaaaggatcaacagctcctggggatttggggttgctctggaaaactcatttgcaccactactgtgccttggaatgctagttggagtaataaatctctgaatgacatttggaataacatgacctggatggagtgggaaagagaaattggcaattacacaggcttaatatacaccttaattgaacaatcacagaaccagcaagaaaagaatgaacaagaattattggaattggataaatgggcaagtttgtggaattggtttgacataacaaaatggttgtggtatataaaaatattca

SEQ ID NO: 5 - protein, 129 aa, Human immunodeficiency virus 1
NLLRAIEAQQHLLQLTVWGIKQLQARILAVERYLQDQQLLGSWGCSGRHICTTNVPWNASWSNKSLDEIWGNMTWIEWEREIDNYTGLIYNLIEESQTQQEKNEQDLLQLDKWASLWNWFSITKWLWYI

SEQ ID NO: 6 - protein, 483 aa, Human immunodeficiency virus 1
SATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 7 - protein, 856 aa, Human immunodeficiency virus 1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 8 - protein, 566 aa, unidentified
MKAKLLILLCTLSATDADTICIGYHANNSTETVDTVLEKNVTVTHSVNLLEDSHNGKLCRLKGITPLQLGKCSIAGWILGNPECESLFSKKSWSYIAETPNPENGICYPGYFSDYEELREQLSSVSSFERFEIFPKESSWPKHSINKGVTASCSHKGKSNFYRNLLWLTEKNGSYPNLSKSYVNDKEKEVLVLWGVHHPSNIEDQRAIYRKETAYVSVVASHYSRRFTPEIARRPKIRDQEGRINYYWTLLGPRDTIIFEANGNLIAPWYAFALSRGFKSGIIISNASMDDCDTKCQTPQGAINSSLPFQSVHPVTIGECPKYVRSTKLRMVTGLRNIPSIQSRGLFGAIAGFIEGGWTGMIDGWYGYHHQNEQGSGYAADQKSTQNAINGITNKVNSVIEKMNTQFTAVGKEFNKLEKRMENLNKKVDDGFLDIWTYNAELLVLLENERTLDFHDSNVKSLYEKVKGQLKNNAKEIGNGCFEFYHKCDNECMDSVKNGTYDYPKYSEESKLNREKIDGVELKSMGVYQILAIYSTVASSLVLLVSLGAISFWMCSNGSLQCRICI

SEQ ID NO: 9 - protein, 560 aa, unidentified
MNTQILVFALIAIIPTNADKICLGHHAVSNGTKVNTLTERGVEVVNATETVERTNIPRICSKGKRTVDLGQCGLLGTITGPPQCDQFLEFSADLIIERREGSDVCYPGKFVNEEALRQILRESGGIDKEAMGFTYSGIRTNGATSACRRSGSSFYAEMKWLLSNTDDAAFPQMTKSYKNTRKSPALIVWGIHHSVSTAEQTKLYGSGNKLVTVGSSNYQQSFVPSPGARPQVNGLSGRIDFHWLMLNPNDTVTFSFNGAFIAPDRASFLRGKSMGIQSGVQVDANCEGDCYHSGGTIISNLPFQNIDSRAVGKCPRYVKQRSLLLATGMKNVPEIPKGRGLFGAIAGFIENGWEGLIDGWYGFRHQNAQGEGTAADYKSTQSAIDQITGKLNRLIEKTNQQFELIDNEFNEVEKQIGNVINWTRDSITEVWSYNAELLVAMENQHTIDLADSEMDKLYERVKRQLRENAEEDGTGCFEIFHKCDDDCMASIRNNTYDHSKYREEAMQNRIQIDPVKLSSGYKDVILWFSFGASCFILLAIVMGLVFICVKNGNMRCTICI

SEQ ID NO: 10 - protein, 18 aa, unidentified
FIEGGWTGMVDGWYGYHH

SEQ ID NO: 11 - protein, 9 aa, unidentified
NFDKLYIWG

SEQ ID NO: 12 - protein, 13 aa, unidentified
SSRISIYWTIVKP

SEQ ID NO: 13 - protein, 4 aa, unidentified
IEDB

SEQ ID NO: 14 - protein, 6 aa, unidentified
HFEKIQ

SEQ ID NO: 15 - DNA, 81 bp, Synthetic construct
  source 1..81: mol_type = other DNA
tcgggcgagtcgtctgnnnnnnnnnnnnnnnnnnnnnnnnnnnnnnnnnnnnnnnnnnnnnnnnnnccgcatcctcctccc

SEQ ID NO: 16 - DNA, 81 bp, Synthetic construct
  source 1..81: mol_type = other DNA
tcgggcgagtcgtctgagctgtcgagtcgaaggttgacgtagcgccgtagcatcgatgcccgtttgccgcatcctcctccc

SEQ ID NO: 17 - DNA, 81 bp, Synthetic construct
  source 1..81: mol_type = other DNA
tcgggcgagtcgtctgagctgtcgagtcgaagcatgacgtagcgccgtagcatcgttgcccgtttgccgcatcctcctccc

SEQ ID NO: 18 - DNA, 81 bp, Synthetic construct
  source 1..81: mol_type = other DNA
tcgggcgagtcgtctgagctgtcgagtttaaggttgacgtagcgccgtagcatcgcctcccgtttgccgcatcctcctccc

SEQ ID NO: 19 - DNA, 81 bp, Synthetic construct
  source 1..81: mol_type = other DNA
tcgggcgagtcgtctgcctagtcgagtcgaaggttgacgtagcgccgtaaggtcgatgcccgtttgccgcatcctcctccc

SEQ ID NO: 20 - DNA, 81 bp, Synthetic construct
  source 1..81: mol_type = other DNA
tcgggcgagtcgtctgagctgtacagtcgaaggttggcgtagcgcctaagcatcgatgagcgtttgccgcatcctcctccc

SEQ ID NO: 21 - DNA, 81 bp, Synthetic construct
  source 1..81: mol_type = other DNA
tcgggcgagtcgtctgagctgacaagtcgaaggttgacgtttcgccgtagcatcgacgcccgtttgccgcatcctcctccc

SEQ ID NO: 22 - DNA, 81 bp, Synthetic construct
  source 1..81: mol_type = other DNA
tcgggcgagtcgtctgagctgacgagtcgaaggttgacgtttcgccgtagcatcgatgccgatttgccgcatcctcctccc

SEQ ID NO: 23 - DNA, 36 bp, Synthetic construct
  source 1..36: mol_type = other DNA
ttttttatcagggctaaagagtgcagagttacttag

SEQ ID NO: 24 - DNA, 60 bp, Synthetic construct
  source 1..60: mol_type = other DNA
agcagcacagaggtcagatggcaggtaagcaggcggctcacaaaaccattcgcatgcggc

SEQ ID NO: 25 - DNA, 80 bp, Synthetic construct
  source 1..80: mol_type = other DNA
gggaatggatccacatctacgaattcatcagggctaaagagtgcagagttacttagttcactgcagacttgacgaagctt

SEQ ID NO: 26 - DNA, 80 bp, Synthetic construct
  source 1..80: mol_type = other DNA
agcagcacagaggtcagatggcaggtaagcaggcggctcacaaaaccattcgcatgcggccctatgcgtgctaccgtgaa

SEQ ID NO: 27 - DNA, 96 bp, Synthetic construct
  source 1..96: mol_type = other DNA
ataccagcttattcaattctatactccactttgctatttctcggttccttcacgcgccgatcgcaggctgatgaattgagatagtaagtgcaatct

SEQ ID NO: 28 - DNA, 40 bp, Synthetic construct
  source 1..40: mol_type = other DNA
gctatgggtggtctggttgggattggccccgggagctggc

(19) United States
(12) Patent Application Publication
(10) Pub. No.: US 2026/0284180 A1
(43) Pub. Date: Sep. 24, 2026

(54) COMPOSITIONS COMPRISING APTAMERS AND METHODS OF MODULATING THE IMMUNE RESPONSE USING THE SAME

(71) Applicant: The Wistar Institute of Anatomy and Biology, Philadelphia, PA (US)

(72) Inventor: Amelia Escolano, Philadelphia, PA (US)

(73) Assignee: The Wistar Institute of Anatomy and Biology, Philadelphia, PA (US)

(21) Appl. No.: 19/477,465

(22) PCT Filed: Apr. 22, 2024
(86) PCT No.: PCT/US2024/025692

§ 371 Date: Oct. 21, 2025

Related U.S. Application Data
(60) Provisional application No. 63/497,486, filed on Apr. 21, 2023.

(51) Int. Cl. A61K 39/215; A61K 39/00

(52) U.S. Cl. CPC A61K 39/215; A61K 2039/53

(57) ABSTRACT

The disclosure relates to compositions comprising aptamers or functional fragments thereof, and methods of using aptamers to associate with an antigen administered to a subject, such that aptamers associate with certain immunodominant epitopes on the antigen and reduce the immunogenicity of the antigen in respect to those immunodominant epitopes.

### CROSS-REFERENCE TO RELATED APPLICATION

This application claims the benefit of U.S. provisional application No. 63/497,486, which was filed Apr. 21, 2023, and entitled “COMPOSITIONS COMPRISING APTAMERS AND METHODS OF MODULATING THE IMMUNE RESPONSE USING THE SAME,” and which is incorporated by reference in its entirety.

### STATEMENT REGARDING FEDERALLY SPONSORED RESEARCH

This invention was made with government support under grant number 1 DP2 AI175470-01 awarded by the National Institutes of Health. The government has certain rights in this invention.

### REFERENCE TO ELECTRONIC SEQUENCE LISTING

The Sequence Listing filed herewith, created on Apr. 22, 2024, having the filename WIST-011-PCT.xml, and having a file size of 35,638 bytes is incorporated by reference in its entirety.

### FIELD

The disclosure relates to compositions comprising aptamers or functional fragments thereof, and methods of using aptamers to associate with an antigen administered to a subject, such that aptamers associate with certain immunodominant epitopes on the antigen and reduce the immunogenicity of the antigen in respect to those immunodominant epitopes.

### BACKGROUND

Infectious diseases are serious and recurrent health threats. Efficacious vaccines to prevent infection by current or newly arising pathogens are highly desirable. Particularly concerning are viruses with the capacity to rapidly mutate. HIV-1, influenza or SARS-COV2 have the ability to mutate to adapt to new hosts and environments, escaping from the pressure exerted by the host immune system. This ability to mutate results in a large diversity of circulating viral variants, characterized by individual antigenic and infectivity properties¹. Efficacious vaccines to broadly protect against all these variants should induce antibodies against their conserved viral epitopes; however, directing antibody responses to conserved epitopes by vaccination is incredibly challenging. Failure to do so results in polyclonal antibody responses to different non-conserved viral epitopes, which severely interfere with the development of broadly protective responses. Very recently, it has been reported that HIV vaccine prototypes being evaluated in early human clinical trials generate antibodies to non-conserved viral epitopes that actively hinder protection by degrading the vaccine's payload². Despite years of research, no universal vaccines are available against highly diverse viruses such as HIV-1 or influenza, and well-founded concerns are being raised about the efficacy of the recently developed vaccines against future variants of SARS-CoV2³.

### SUMMARY

The disclosure relates to a method of vaccinating a subject administering a composition comprising one or more one or more aptamers targeting one or a plurality of immunodominant epitopes of one or more antigens. In some embodiments, the composition further comprises administering: (i) one or more antigens or nucleic acid sequences encoding the one or more antigens comprising an immunodominant epitope. In some embodiments, the methods further comprise administering to a subject in need thereof: (i) a nucleic acid sequence encoding one or a plurality of adjuvants; or (ii) one or a plurality of adjuvants. In some embodiments, the one or more adjuvants are chosen from IL-12, IL-2, RANTES, MIP-1alpha, IL-8, IL-15, IL-17, IL-28, GM-CSF, IL-15, IL-21, IL-23, soluble LAG3, agonist CD28, anti-PD1, anti-PDL1/2, anti-OX40/OX40L, anti-GITR/GITRL, and/or anti-TIM3.

The disclosure relates to administration and elicitation of immune responses against antigens. In some embodiments, the antigen is a viral antigen, such as a lentiviral antigen, SARS antigen, or influenza antigen.

The disclosure also relates to the preparation and/or manufacture of compositions comprising aptamers that target and bind to immunodominant epitopes on the antigens. In some embodiments, the immunodominant epitopes are fold 1 and fold 2 of gp120; or fold 1 or 2 of gp41 or a fragment thereof that comprises at least about 70% sequence identity to the fold 1 and fold 2 of gp 120; or at least about 70% sequence identity to the fold 1 or 2 of gp41.

In some embodiments, the aptamer comprises an IC₅₀ of the association to the immunodominant epitopic from about 10 nanomolar to about 1.5 micromolar. In some embodiments, the aptamer comprises an IC₅₀ of the association to the immunodominant epitope from about 10 nM to about 500 nM micromolar. In some embodiments, the aptamer binds to or associates with the epitope comprising at least about 90% sequence identity to a SARS-COV2 epitope, HIV-1 epitope, or influenza epitope disclosed in Tables X or Y.

The disclosure relates to a method of enhancing the immunogenicity of one or a plurality of epitopes of an antigen in a subject comprising administering to the subject in need thereof an aptamer capable of associating with one or a plurality of immunodominant epitopes of the antigen.

The disclosure relates to a method of neutralizing viral infection or reducing viral load in a subject in need thereof comprising administering to the subject in need thereof an aptamer capable of associating with one or a plurality of immunodominant epitopes of an antigen.

The disclosure also relates to a method of reducing the immunogenicity of immunodominant epitopes of antigens, such as viral antigens, by administering a therapeutically effective amount of an aptamer that binds to or associate with the immunodominant epitope. The disclosure relates to pharmaceutical compositions comprising a therapeutically effective amount of any of the disclosed aptamer sequences; and a pharmaceutically acceptable carrier.

### BRIEF DESCRIPTION OF DRAWINGS

FIG. 1 depicts a graphical representation of aptamer strategy. FIG. 1A, Graphical representation of an aptamer-antigen interaction. FIG. 1B, Diagram describing the SELEX method for aptamer selection.

FIGS. 2A and 2B depict a representation of how aptamers may characterize antibody responses. Using antigen-aptamer complexes to classify B cells according to their antigen specificities. FIG. 2C depicts using aptamers for epitope mapping.

FIG. 3 depicts a schematic of data demonstrating that aptamers bind to their specific antigens and mask epitopes. 3A, ELISA results showing specific binding of an aptamer to HEL. 3B, BLI assay showing specific binding of an aptamer to a SAV-coated sensor. 3C, BLI assay in 3D. D, BLI assay showing competition between a SAV aptamer and a biotinylated SARS-RBD protein for binding to SAV. Lower association of biot-SARS-RBD is observed in the presence of SAV aptamer. Moreover, a 2-phase dissociation curve is observed in the presence of SAV aptamer indicating that two species are bound to the sensor: 1) Aptamer (fast dissociation), and 2) biot-SARS-RBD (no dissociation). The amount of biot-SARS-RBD bound to the aptamer-coated sensor is lower.

FIG. 4 depicts how aptamers can be internalized by B cells through the BCR and can be detected inside the cell. 4A, Graphical representation of the method used to evaluate BCR-mediated internalization of a random DNA oligonucleotide by NP-specific B cells. 4B, Flow cytometry analysis of NP-specific B cells stimulated for 30 minutes as indicated on the figure. 4C, Graphical representation of the method used to confirm aptamer internalization through BCR. 4D, Flow cytometry plot showing fluorescein and kappa light chain (Ig Kappa) expression in Env-specific B cells, 2 hours after receiving the indicated stimuli for 30 min.

### DETAILED DESCRIPTION OF EMBODIMENTS

Before the present compositions and methods are described, it is to be understood that this disclosure is not limited to the particular molecules, compositions, methodologies or protocols described, as these may vary. It is also to be understood that the terminology used in the description is for the purpose of describing the particular versions or embodiments only, and is not intended to limit the scope of the present disclosure which will be limited only by the appended claims. It is understood that these embodiments are not limited to the particular methodology, protocols, cell lines, vectors, and reagents described, as these may vary. It also is to be understood that the terminology used herein is for the purpose of describing particular embodiments only, and is not intended to limit the scope of the present embodiments or claims.

Unless defined otherwise, all technical and scientific terms used herein have the same meanings as commonly understood by one of ordinary skill in the art. Although any methods and materials similar or equivalent to those described herein can be used in the practice or testing of embodiments of the present disclosure, the preferred methods, devices, and materials are now described. All publications mentioned herein are incorporated by reference. Nothing herein is to be construed as an admission that the disclosure is not entitled to antedate such disclosure by virtue of prior disclosure.

It must be noted that as used herein and in the appended claims, the singular forms “a”, “an,” and “the” include plural reference unless the context clearly dictates otherwise. Thus, for example, reference to “a nucleic acid sequence” includes a plurality of nucleotides that are formed, reference to “the nucleic acid sequence” is a reference to one or more nucleic acid sequences and equivalents thereof known to those skilled in the art, and so forth.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. The term “about” as used herein when referring to a measurable value such as an amount, a temporal duration, and the like, is meant to encompass variations of ±20%, ±10%, ±5%, ±1%, ±0.5%, or ±0.1% from the specified value, as such variations are appropriate to perform the disclosed methods.

As used herein, the terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” in context of an immunotherapy refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, indirect or direct ligation of cell surface moieties, even in the absence of a direct signal transduction event, may result in the reorganization of cytoskeletal structures, or in the coalescing of cell surface moieties, each of which could serve to enhance, modify, or alter subsequent cellular responses. As used herein, the terms “activating CD8+ T cells” or “CD8+ T cell activation” refer to a process (e.g., a signaling event) causing or resulting in one or more cellular responses of a CD8+ T cell (CTL), selected from: proliferation, differentiation, cytokine secretion, cytotoxic effector molecule release, cytotoxic activity, and expression of activation markers. As used herein, an “activated CD8+ T cell” refers to a CD8+ T cell that has received an activating signal, and thus demonstrates one or more cellular responses, selected from proliferation, differentiation, cytokine secretion, cytotoxic effector molecule release, cytotoxic activity, and expression of activation markers. Suitable assays to measure CD8+ T cell activation are known in the art and are described herein. In the disclosed methods the administration of an aptamer is free of a biological elicitation of an immune response against the aptamer. In other words, in some embodiments, the subject does not comprise activated CD8+ T cells against the administered aptamer. As used herein, the terms “activating B cells” or “B cell activation” refer to a process (e.g., a signaling event) causing or resulting in one or more cellular responses of B cell, selected from: proliferation, differentiation, antibody secretion, and expression of activation markers. As used herein, an “activated B cell” refers to a B cell that has received an activating signal, and thus demonstrates one or more cellular responses, selected from proliferation, clonal expansion and/or antibody secretion. Suitable assays to measure B cell activation are known in the art and are described herein. In the disclosed methods the administration of an aptamer is free of a biological elicitation of an immune response against the aptamer. In other words, in some embodiments, the subject does not comprise activated B cells secreting measurable amount or biologically effective amount of antibody against the administered aptamer.

The term “combination therapy” as used herein is meant to refer to administration of one or more therapeutic agents in a sequential manner, that is, wherein each therapeutic agent is administered at a different time; as well as administration of these therapeutic agents, or at least two of the therapeutic agents, in a substantially simultaneous manner. In some embodiments, the therapeutic agents are administered within 1, 2, 3, 4, 5, 6, 7, 8, 9, or 10 minutes within each other. Substantially simultaneous administration can be accomplished, for example, by administering to the subject a single dose having a fixed ratio of each therapeutic agent or in multiple, individual doses for each of the therapeutic agents. For example, one combination of the present disclosure may comprise a pooled sample of one or more nucleic acid molecules comprising one or a plurality of expressible nucleic acid sequences and an adjuvant and/or an anti-viral agent administered at the same or different times. In some embodiments, the pharmaceutical composition of the disclosure can be formulated as a single, co-formulated pharmaceutical composition comprising one or more nucleic acid molecules comprising one or a plurality of expressible nucleic acid sequences and one or more adjuvants and/or one or more anti-viral agents. As another example, a combination of the present disclosure (e.g., DNA or RNA vaccines and anti-viral agent) may be formulated as separate pharmaceutical compositions that can be administered at the same or different time. As used herein, the term “simultaneously” is meant to refer to administration of one or more agents at the same time. For example, in certain embodiments, antiviral vaccine or immunogenic composition and antiviral agents are administered simultaneously). Simultaneously includes administration contemporaneously or immediately sequentially, that is during the same period of time. In certain embodiments, the one or more agents are administered simultaneously in the same hour, or simultaneously in the same day. Sequential or substantially simultaneous administration of each therapeutic agent can be effected by any appropriate route including, but not limited to, oral routes, intravenous routes, sub-cutaneous routes, intramuscular routes, direct absorption through mucous membrane tissues (e.g., nasal, mouth, vaginal, and rectal), and ocular routes (e.g., intravitreal, intraocular, etc.). The therapeutic agents can be administered by the same route or by different routes. For example, one component of a particular combination may be administered by intravenous injection while the other component(s) of the combination may be administered intramuscularly only. The components may be administered in any therapeutically effective sequence. A “combination” embraces groups of compounds or non small chemical compound therapies useful as part of a combination therapy. In some embodiments, the therapeutic agent is an anti-retroviral therapy, (such as one or a combination of efavirenz, lamivudine and tenofovir disoproxil fumarate) or anti-flu therapy (such as TamiFlu®). In some embodiments, the therapeutic agent is one or a combination of: abacavir/dolutegravir/lamivudine (Triumeq), dolutegravir/rilpivirine (Juluca), elvitegravir/cobicistat/emtricitabine/tenofovir disoproxil fumarate (Stribild), elvitegravir/cobicistat/emtricitabine/tenofovir alafenamide (Genvoya), efavirenz/emtricitabine/tenofovir disoproxil fumarate (Atripla), emtricitabine/rilpivirine/tenofovir disoproxil fumarate (Complera), emtricitabine/rilpivirine/tenofovir alafenamide (Odefsey), bictegravir, emtricitabine, and tenofovir alafenamide (Biktarvy). In some embodiments, the therapeutic agent is one or a combination of a reverse transcrioptase inhibitor of a retrovirus such as efavirenz (Sustiva), etravirine (Intelence), nevirapine (Viramune), nevirapine extended-release (Viramune XR), rilpivirine (Edurant), delavirdine mesylate (Rescriptor). In some embodiments, the therapeutic agent is one or a combination of a protease inhibitor of a retrovirus, such as: atazanavir/cobicistat (Evotaz), darunavir/cobicistat (Prezcobix), lopinavir/ritonavir (Kaletra), ritonavir (Norvir), atazanavir (Reyataz), darunavir (Prezista), fosamprenavir (Lexiva), tipranavir (Aptivus).

As used herein, “expression” refers to the process by which a polynucleotide is transcribed from a DNA template (such as into and mRNA or other RNA transcript) and/or the process by which a transcribed mRNA (or administered mRNA) is translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. In some embodiments, the at least one expressible nucleic acid sequence comprises only DNA nucleotides, RNA nucleotides or comprises both RNA and DNA nucleotides. In some embodiments, the at least one expressible nucleic acid consists of RNA. In some embodiments, the at least one expressible nucleic acid consists of DNA. In some embodiments, the expressible nucleic acid sequence, also known as a coding sequence, encodes one or a plurality of immunodominant epitopes and/or one or a plurality of immunotherapeutic epitopes of an antigen. In some embodiments, the antigen is a viral antigen.

The terms “functional fragment” means any portion of a polypeptide or nucleic acid sequence from which the respective full-length polypeptide or nucleic acid relates that is of a sufficient length and has a sufficient structure to confer a biological affect that is at least similar or substantially similar to the full-length polypeptide or nucleic acid upon which the fragment is based. In some embodiments, a functional fragment is a portion of a full-length or wild-type nucleic acid sequence that encodes any one of the nucleic acid sequences disclosed herein, and said portion encodes a polypeptide of a certain length and/or structure that is less than full-length but encodes a domain that still biologically functional as compared to the full-length or wild-type protein. In some embodiments, the functional fragment may have a reduced biological activity, about equivalent biological activity, or an enhanced biological activity as compared to the wild-type or full-length polypeptide sequence upon which the fragment is based (such wild-type or full-length sequences “reference sequences” or each individually a “reference sequence”). In some embodiments, the functional fragment is derived from the sequence of an organism, such as a human. In such embodiments, the functional fragment may retain about 99%, 98%, 97%, 96%, 95%, 94%, 93%, 92%, 91%, or 90% sequence identity to the wild-type human sequence upon which the sequence is derived. In some embodiments, the functional fragment may retain about 85%, 80%, 75%, 70%, 65%, or 60% sequence identity to the wild-type sequence upon which the sequence is derived.

By “fragment” is meant a portion of a polypeptide or nucleic acid molecule. This portion contains, preferably, at least about 5%, 10%, 20%, 30%, 40%, 50%, 60%, 70%, 80%, or about 90% of the entire length of the reference nucleic acid molecule or polypeptide. A fragment may contain about 5, 10, 20, 30, 40, 50, 60, 70, 80, 90, 100, 200, 300, 400, 500, 600, 700, 800, 900, 1000 or more nucleotides or amino acids.

“Optional” or “optionally” means that the subsequently described event, circumstance, or material may or may not occur or be present, and that the description includes instances where the event, circumstance, or material occurs or is present and instances where it does not occur or is not present.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in some embodiments, to A without B (optionally including elements other than B); in another embodiments, to B without A (optionally including elements other than A); in yet another embodiments, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of.” “Consisting essentially of,” when used in the claims, shall have its ordinary meaning as used in the field of patent law.

As used herein an “antigen” is meant to refer to any substance that elicits an immune response.

As used herein, the term “electroporation,” “electro-permeabilization,” or “electro-kinetic enhancement” (“EP”), are used interchangeably and are meant to refer to the use of a transmembrane electric field pulse to induce microscopic pathways (pores) in a bio membrane; their presence allows biomolecules such as plasmids, oligonucleotides, siRNA, drugs, ions, and/or water to pass from one side of the cellular membrane to the other. In some of the disclosed methods of treatment or prevention, the method comprises a step of electroporation of a subject's tissue for a sufficient time and with a sufficient electrical field capable of inducing uptake of the pharmaceutical compositions disclosed herein into the antigen-presenting cells. In some embodiments, the cells are antigen presenting cells. In some embodiments, the cells are B cells.

The term “pharmaceutically acceptable excipient,” “pharmaceutically acceptable carrier” or “pharmaceutically acceptable diluent” as used herein is meant to refer to an excipient, carrier or diluent that can be administered to a subject, together with an agent or the pharmaceutical compositions disclosed herein, and which is inert or fails to eliminate the pharmacological activity of the active agent of the pharmaceutical composition. In some embodiments, the pharmaceutically acceptable carrier does fails to destroy or is incapable of eliminating the pharmacological activity of an active agent/vaccine and is nontoxic when administered in doses sufficient to deliver a therapeutic amount of the active agent. The term “pharmaceutically acceptable salt” of nucleic acids as used herein may be an acid or base salt that is generally considered in the art to be suitable for use in contact with the tissues of human beings or animals without excessive toxicity, irritation, allergic response, or other problem or complication. Such salts include mineral and organic acid salts of basic residues such as amines, as well as alkali or organic salts of acidic residues such as carboxylic acids. Specific pharmaceutical salts include, but are not limited to, salts of acids such as hydrochloric, phosphoric, hydrobromic, malic, glycolic, fumaric, sulfuric, sulfamic, suifanilic, formic, toluenesulfonie, methanesulfonic, benzene sulfonic, ethane disulfonic, 2-hydroxyethyl sulfonic, nitric, benzoic, 2-acetoxybenzoic, citric, tartaric, lactic, stearic, salicylic, glutamic, ascorbic, pamoic, succinic, fumaric, maleic, propionic, hydroxymaleic, hydroiodic, phenylacetic, alkanoic such as acetic, HOOC-(CH2)n-COOH where n is 0-4, and the like. Similarly, pharmaceutically acceptable cations include, but are not limited to sodium, potassium, calcium, aluminum, lithium and ammonium. Those of ordinary skill in the art will recognize from this disclosure and the knowledge in the art that further pharmaceutically acceptable salts for the pooled viral specific antigens or polynucleotides provided herein, including those listed by Remington's Pharmaceutical Sciences, 17th ed., Mack Publishing Company, Easton, PA, p. 1418 (1985). In general, a pharmaceutically acceptable acid or base salt can be synthesized from a parent compound that contains a basic or acidic moiety by any conventional chemical method. Briefly, such salts can be prepared by reacting the free acid or base forms of these compounds with a stoichiometric amount of the appropriate base or acid in an appropriate solvent.

As used herein, the terms “prevent,” “preventing,” “prevention,” “prophylactic treatment,” and the like, are meant to refer to reducing the probability of developing a disease or condition in a subject, who does not have, but is at risk of or susceptible to developing a disease or condition.

As used herein, the term “purified” means that the polynucleotide or polypeptide or fragment, variant, or derivative thereof is substantially free of other biological material with which it is naturally associated, or free from other biological materials derived, e.g., from a recombinant host cell that has been genetically engineered to express the polypeptide of the present disclosure. That is, e.g., a purified polypeptide of the present disclosure is a polypeptide that is at least from about 70 to 100% pure, i.e., the polypeptide is present in a composition wherein the polypeptide constitutes from about 70 to about 100% by weight of the total composition. In some embodiments, the purified polypeptide of the present disclosure is from about 75% to about 99% by weight pure, from about 80% to about 99% by weight pure, from about 90 to about 99% by weight pure, or from about 95% to about 99% by weight pure.

The terms “subject,” “individual,” and “patient” are used interchangeably herein to refer to a vertebrate, preferably a mammal, more preferably a human. Mammals include, but are not limited to, murine, simians, humans, farm animals, cows, pigs, goats, sheep, horses, dogs, sport animals, and pets. Tissues, cells and their progeny obtained in vivo or cultured in vitro are also encompassed by the definition of the term “subject.” The term “subject” is also used throughout the specification in some embodiments to describe an animal from which a cell sample is taken or an animal to which a disclosed cell or nucleic acid sequences have been administered. In some embodiment, the subject is a human. For treatment of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a non-human animal. The term “mammal” encompasses both humans and non-humans and includes but is not limited to humans, non-human primates, canines, felines, murine, bovines, equines, caprine, and porcines. In some embodiments, the mammal is a donkey, camel, rabbit, guinea pig, horse, pig, cow, cat, dog, rat, mouse, monkey, non-human, ape or human.

The term “therapeutic effect” as used herein is meant to refer to some extent of relief of one or more of the symptoms of a disorder (e.g., viral infection) or its associated pathology. A “therapeutically effective amount” as used herein is meant to refer to an amount of an agent which is effective, upon single or multiple dose administration (such as a first, second and/or third booster) to the cell or subject, in prolonging the survivability of the patient with such a disorder, reducing one or more signs or symptoms of the disorder, preventing or delaying, and the like beyond that expected in the absence of such treatment. A “therapeutically effective amount” is intended to qualify the amount of a substance or composition required to achieve a therapeutic effect. A physician or veterinarian having ordinary skill in the art can readily determine and prescribe the “therapeutically effective amount” (e.g., K_{D}, ED₅₀ or IC₅₀) of the pharmaceutical composition required. For example, the physician or veterinarian could start doses of the compounds of the present disclosure employed in a pharmaceutical composition at levels lower than that required in order to achieve the desired therapeutic effect and gradually increase the dosage until the desired effect is achieved.

The terms “treat,” “treated,” “treating,” “treatment,” and the like as used herein are meant to refer to reducing or ameliorating a disorder and/or symptoms associated therewith (e.g., a viral infection). “Treating” can refer to administration of the DNA and/or RNA vaccines described herein to a subject after the onset, or suspected onset, of a viral infection. “Treating” includes the concepts of “alleviating,” which refers to lessening the frequency of occurrence or recurrence, or the severity, of any symptoms or other ill effects related to a virus and/or the side effects associated with viral therapy. The term “treating” also encompasses the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated.

For any therapeutic agent described herein the therapeutically effective amount may be initially determined from preliminary in vitro studies and/or animal models. A therapeutically effective dose may also be determined from human data. The applied dose can be adjusted based on the relative bioavailability and potency of the administered agent. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below. Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained. Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The terms “polynucleotide,” “oligonucleotide” and “nucleic acid” are used interchangeably throughout and include DNA molecules (e.g., cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. The nucleic acid molecule can be single-stranded or double-stranded. In some embodiments, the nucleic acid molecules of the disclosure comprise a contiguous open reading frame encoding an antibody, or a fragment thereof, as described herein. “Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions or may associate with an antigen. Thus, a nucleic acid also encompasses an aptamer that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. A nucleic acid will generally contain phosphodiester bonds, although nucleic acid analogs maybe included that may have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5,034,506, which are incorporated by reference in their entireties.

Nucleic acids containing one or more non-naturally occurring or modified nucleotides are also included within one definition of nucleic acids. The modified nucleotide analog may he located for example at the 5′-end and/or the 3′-end of the nucleic acid molecule. Representative examples of nucleotide analogs may be selected from sugar- or backbone-modified ribonucleotides. It should be noted, however, that also nucleobase-modified ribonucleotides, i.e. ribonucleotides, containing a non-naturally occurring nucleobase instead of a naturally occurring nucleobase such as uridines or cytidines modified at the 5-position, e.g. 5-(2-amino) propyl uridine, 5-bromo uridine; adenosines and guanosines modified at the 8-position, e.g. 8-bromo guanosine; deaza nucleotides, e.g. 7-deaza-adenosine; 0- and N-alkylated nucleotides, e.g. N6-methyl adenosine are suitable. The 2′-OH-group may be replaced by a group selected from H, OR, R, halo, SH, SR, NTh, NHR, N2 or CN, wherein R is C1-C6 alkyl, alkenyl or alkynyl and halo is F, Cl, Br or I. Modified nucleotides also include nucleotides conjugated with cholesterol through, e.g., a hydroxyprolinol linkage as described in Krutzfeldt et ak, Nature (Oct. 30, 2005), Soutschek et ak, Nature 432:173-178 (2004), and U.S. Patent Publication No. 20050107325, which are incorporated herein by reference in their entireties. Modified nucleotides and nucleic acids may also include locked nucleic acids (LNA), as described in US20020115080, which is incorporated herein by reference. Additional modified nucleotides and nucleic acids are described in U.S. Patent Publication No. 20050182005, which is incorporated herein by reference in its entirety. Modifications of the ribose-phosphate backbone may be done for a variety of reasons, e.g., to increase the stability and half-life of such molecules in physiological environments, to enhance diffusion across cell membranes, or as probes on a biochip. Mixtures of naturally occurring nucleic acids and analogs may be made; alternatively, mixtures of different nucleic acid analogs, and mixtures of naturally occurring nucleic acids and analogs may be made. In some embodiments, the expressible nucleic acid sequence is in the form of DNA. In some embodiments, the expressible nucleic acid is in the form of RNA with a sequence that encodes the polypeptide sequences disclosed herein and, in some embodiments, the expressible nucleic acid sequence is an RNA/DNA hybrid molecule that encodes any one or plurality of polypeptide sequences disclosed herein.

As used herein, the term “nucleic acid molecule” is a molecule that comprises one or more nucleotide sequences that encode one or more proteins. In some embodiments, a nucleic acid molecule comprises initiation and termination signals operably linked to regulatory elements including a promoter and polyadenylation signal capable of directing expression in the cells of the individual to whom the nucleic acid molecule is administered. In some embodiments, the nucleic acid molecule also includes a plasmid containing one or more nucleotide sequences that encode one or a plurality of viral antigens. In some embodiments, the disclosure relates to a pharmaceutical composition comprising a first, second, third or more nucleic acid molecule, each of which encoding one or a plurality of viral antigens and at least one of each plasmid comprising one or more of the compositions disclosed herein.

The terms “polypeptide,” “peptide” and “protein” are used interchangeably herein to refer to polymers of amino acids of any length. The polymer may be linear or branched, it may comprise modified amino acids, and it may be interrupted by non-natural amino acids or chemical groups that are not amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. In some embodiments, the polypeptide is an HIV-1 antigen, COVID antigen, or influenza antigen. In some embodiments, the disclosure comprises a composition comprising an aptamer that is specific for an immunodominant epitope that is not an immunotherapeutic epitope on an antigen.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide can be complementary to another polynucleotide without being its complement.

The term “hybridization” or “hybridizes” as used herein refers to the formation of a duplex between nucleotide sequences that are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T (U), G and C of one sequence is then aligned with a T (U), A, C and G, respectively, of the other sequence. RNA sequences can also include complementary G=U or U=G base pairs. Thus, two sequences need not have perfect homology to be “complementary.” Usually, two sequences are sufficiently complementary when at least about 90% (preferably at least about 95%) of the nucleotides share base pair organization over a defined length of the molecule.

By “substantially identical” is meant nucleic acid molecule (or polypeptide) exhibiting at least about 50% identity to a reference amino acid sequence (for example, any one of the amino acid sequences described herein) or nucleic acid sequence (for example, any one of the nucleic acid sequences described herein). In some embodiments, such a sequence is at least about 60%, 70%, 80% or 85%, 90%, 95% or even 99% identical at the amino acid level or nucleic acid to the sequence used for comparison.

A nucleotide sequence is “operably linked” to a regulatory sequence if the regulatory sequence affects the expression (e.g., the level, timing, or location of expression) of the nucleotide sequence. A “regulatory sequence” is a nucleic acid that affects the expression (e.g., the level, timing, or location of expression) of a nucleic acid to which it is operably linked. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif and Baron et al., 1995, Nucleic Acids Res. 23:3605-06.

As used herein, “specific for” or “specifically binds to” means that the binding affinity of an antibody to an antigen or a ligand to a ligand-binding partner is statistically higher than the binding affinity of the same substrate to a generally comparable, but off-target amino acid sequence. Normally, the binding affinity of a substrate to a specified target amino acid sequence is at least 1.5 fold, and preferably 2 fold or 5 fold, of the binding affinity of the same substrate to a non-target amino acid sequence. It also refers to binding of a substrate to a specified nucleic acid target sequence to a detectably greater degree, e.g., at least about 1.5-fold over background, than its binding to non-target nucleic acid sequences and to the substantial exclusion of non-target nucleic acids. The substrate's K_{d} or IC₅₀ to each amino acid sequence can be compared to assess the binding specificity of the substrate to a particular target amino acid sequence. In some embodiments, the one or plurality of disclosed aptamers are specific for, or specifically binds, to an antigen, such as a viral antigen or a cancer antigen at an immunodominant epitope that is not immunotherapeutic.

The disclosed compositions comprising aptamer elicit an immune response that leads to secretion of an antibody. The term “antibody” as used herein refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. As used herein, a “targeted binding agent” is an antibody, or binding fragment thereof, that preferentially binds to a target site such as an immunotherapeutic epitope of an antigen. In one embodiment, the targeted binding agent is specific for only one target site. In other embodiments, the targeted binding agent is specific for more than one target site. In one embodiment, the targeted binding agent may be a monoclonal antibody and the target site may be an epitope. “Epitope” refers to that portion of an antigen or other macromolecule capable of forming a binding interaction that interacts with the variable region binding pocket and/or at least one CDR of an antibody. “Binding fragments” of an antibody are produced by recombinant DNA techniques, or by enzymatic or chemical cleavage of intact antibodies. Binding fragments include Fab, Fab′, F(ab′)2, Fv, and single-chain antibodies. An antibody other than a “bispecific” or “bifunctional” antibody is understood to have each of its binding sites identical. An antibody substantially inhibits adhesion of a receptor to a counter-receptor when an excess of antibody reduces the quantity of receptor bound to counter-receptor by at least about 20%, 40%, 60% or 80%, and more usually greater than about 85% (as measured in an in vitro competitive binding assay). An antibody may be oligoclonal, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody and antibodies that can be labeled in soluble or bound form as well as fragments, variants or derivatives thereof, either alone or in combination with other amino acid sequences provided by known techniques. An antibody may be from any species. The term antibody also includes binding fragments of the antibodies of the invention; exemplary fragments include Fv, Fab, Fab′, single stranded antibody (svFC), dimeric variable region (Diabody) and di-sulfide stabilized variable region (dsFv). As discussed herein, minor variations in the amino acid sequences of antibodies or immunoglobulin molecules are contemplated as being encompassed by the present invention, providing that the variations in the amino acid sequence maintain at least about 75%, at least about 80%, at least about 90%, at least about 95%, and about 99% sequence identity to the antibodies or immunoglobulin molecules described herein. In particular, conservative amino acid replacements are contemplated. Conservative replacements are those that take place within a family of amino acids that have related side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are an aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding function or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. In some embodiments, amino- and carboxy-termini of fragments or analogs occur near boundaries of functional domains. Structural and functional domains can be identified by comparison of the nucleotide and/or amino acid sequence data to public or proprietary sequence databases. In some embodiments the CDR or CDR functional fragments binds or associates to an immunotherapeutic epitope of an antigen, such as a viral antigen and is free of a CDR that binds or associates to an immunodominant epitope of the same viral antigen. Preferably, computerized comparison methods are used to identify sequence motifs or predicted protein conformation domains that occur in other proteins of known structure and/or function. Methods to identify protein sequences that fold into a known three-dimensional structure are known See, for example, Bowie et al. Science 253:164 (1991), which is incorporated by reference in its entirety.

An “immunodominant epitope” means an epitope of an antigen that stimulates or induces the majority of immune responses when the antigen is administered to a subject, such that the immune response is skewed in favor of the immunodominant epitope rather than other non-immunodominant epitopes. In some embodiments, while the antigen is a viral antigen, the immunodominant epitope is free of all or a part of the immunotherapeutic epitope, such that administration of the viral antigen results in an immune response skewed for the immunodominant epitope that fails to protect the subject from challenge with second or continued exposure to the viral antigen or fails to effectively mount a therapeutic effect in a subject infected by the virus from which the antigen is derived. It is understood that an immune response generated against immunodominant epitope does not necessarily mean that an immune response against an immunotherapeutic epitope is not stimulated, only that the number of B cells or T cells that recognize the immunotherapeutic epitope are insufficient to treat or prevent infection of a pathogen effectively, or effectively treat or prevent disease progression of hyperproliferative disorder.

An “immunotherapeutic epitope” means an epitope, or portion, of an antigen that, when exposed to a subject, stimulates or induces an immune response that at a certain magnitude can effectively treat or prevent infection of a pathogen, or effectively treat or prevent disease progression of hyperproliferative disorder. In some embodiments, the immunotherapeutic epitope of the disclosure is free of an immunodominant epitope or a portion of an immunodominant epitope.

A “vector” is a molecular vehicle, such as a nucleic acid or protein or proteins, that can be used to introduce or deliver another nucleic acid or protein into a cell. One non-limiting example of vector is a “plasmid,” which refers to a linear or circular double stranded DNA molecule into which additional nucleic acid segments can be ligated. Another type of vector is a viral vector (e.g., replication defective retroviruses, adenoviruses and adeno-associated viruses), comprising additional, exogenous DNA, RNA or hybrid DNA or RNA molecules that can be introduced into the viral genome. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can direct the expression of a chosen polynucleotide. The disclosure relates to any one or plurality of vectors that comprise nucleic acid sequences encoding any one or plurality of amino acid sequence disclosed herein. The disclosure also relates to a vector such as a viral vector comprising a chosen amino acid, wherein such amino acid or a fragment thereof becomes an epitope recognized by a subject's immune system when that vector is administered to the subject. In some embodiments, the expression vector includes from about 30 to about 100,000 nucleotides (e.g., from about 30 to about 50, from about 30 to about 100, from about 30 to about 250, from about 30 to about 500, from about 30 to about 1,000, from about 30 to about 1,500, from about 30 to about 3,000, from about 30 to about 5,000, from about 30 to about 7,000, from about 30 to about 10,000, from about 30 to about 25,000, from about 30 to about 50,000, from about 30 to about 70,000, from about 100 to about 250, from about 100 to about 500, from about 100 to about 1,000, from about 100 to about 1,500, from about 100 to about 3,000, from about 100 to about 5,000, from about 100 to about 7,000, from about 100 to about 10,000, from about 100 to about 25,000, from about 100 to about 50,000, from about 100 to about 70,000, from about 100 to about 100,000, from about 500 to about 1,000, from about 500 to about 1,500, from about 500 to about 2,000, from about 500 to about 3,000, from about 500 to about 5,000, from about 500 to about 7,000, from about 500 to about 10,000, from about 500 to about 25,000, from about 500 to about 50,000, from about 500 to about 70,000, from about 500 to about 100,000, from about 1,000 to about 1,500, from about 1,000 to about 2,000, from about 1,000 to about 3,000, from about 1,000 to about 5,000, from about 1,000 to about 7,000, from about 1,000 to about 10,000, from about 1,000 to about 25,000, from about 1,000 to about 50,000, from about 1,000 to about 70,000, from about 1,000 to about 100,000, from about 1,500 to about 3,000, from about 1,500 to about 5,000, from about 1,500 to about 7,000, from about 1,500 to about 10,000, from about 1,500 to about 25,000, from about 1,500 to about 50,000, from about 1,500 to about 70,000, from about 1,500 to about 100,000, from about 2,000 to about 3,000, from about 2,000 to about 5,000, from about 2,000 to about 7,000, from about 2,000 to about 10,000, from about 2,000 to about 25,000, from about 2,000 to about 50,000, from about 2,000 to about 70,000, and from about 2,000 to about 100,000 nucleotides). In some embodiments, the vector is an attenuated viral vector, such as a lentivirus or an AAV vector that is used to deliver an antigen or a nucleic acid sequence encoding an antigen to a subject. In some embodiments, the vector is an attenuated viral vector, such as a lentivirus or an AAV vector that is used to deliver an aptamer, reverse complement of the aptamer or functional fragments thereof to a subject.

As used herein, the term “kit” refers to a set of components provided in the context of a system for delivering materials to a cell or a subject. Such delivery systems may include, for example, systems that allow for storage, transport, or delivery of various diagnostic or therapeutic reagents (e.g., oligonucleotides, enzymes, extracellular matrix components etc. in appropriate containers) and/or supporting materials (e.g., buffers, media, cells, written instructions for performing the assay etc.) from one location to another. For example, in some embodiments, kits include one or more enclosures (e.g., boxes) containing relevant reaction reagents and/or supporting materials. As used herein, the term “fragmented kit” refers to a diagnostic assay comprising two or more separate containers that each contain a subportion of total kit components. Containers may be delivered to an intended recipient together or separately. For example, a first container may contain a petri dish or polystyrene plate for use in a cell culture assay, while a second container may contain cells, such as control cells. As another example, the kit may comprise a first container comprising a composition comprising one or more disclosed aptamers of the disclosure and, optionally, a second container comprising any one or plurality of reagents necessary for the elicitation of an immune response in an animal against an immunotherapeutic epitope of an antigen. In some embodiments, the second container may comprise an antigen comprising the immunotherapeutic epitope or a nucleic acid sequence encoding the same. In some embodiments, the kit comprises at least one container comprising a composition comprising one or a plurality of the aptamers disclosed herein and a second container comprising reagents needed for isolation of B-cells in cell culture. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contain a sub-portion of total kit components are included in the term “fragmented kit.” In contrast, a “combined kit” refers to a delivery system containing all components in a single container (e.g., in a single box housing each of the desired components). The term “kit” includes both fragmented and combined kits. In some embodiments, the kit of the disclosure comprises an aptamer or a functional fragment thereof, optionally formulated within a nanoparticle or lyophilized and instructions for administering the same.

As used herein, “cell culture” means growth, maintenance, transfection, or propagation of cells, tissues, or their products. As used herein, “culture medium” refers to any solution capable of sustaining the growth of the targeted cells either in vitro or in vivo, or any solution with which targeted cells or exogenous nucleic acids are mixed before being applied to cells in vitro or to a patient in vivo. The disclosure relates to methods of identifying an immunotherapeutic epitope or a generating an immune response against an immunotherapeutic epitope comprising: exposing a cell to an antigen comprising an immunotherapeutic epitope in the presence of an aptamer that is specific for an immunodominant epitope on the antigen. In some embodiments, the step of exposing the cell is performed in vitro in cell culture such that an antigen comprising the immunotherapeutic epitope is exposed to an antigen presenting cell in cell culture under conditions sufficient to propagate the antigen presenting cells and generate an immune response against the immunotherapeutic epitope.

As used herein, the phrase “in need thereof means that the animal or mammal has been identified or suspected as having a need for the particular method or treatment. In some embodiments, the identification can be by any means of diagnosis or observation. In any of the methods and treatments described herein, the animal or mammal can be in need of treatment for viral infection or cancer or in need of treatment for prevention of viral infection or malignant hyperproliferative disease. In some embodiments, the animal or mammal is in an environment or will be traveling to an environment in which a particular disorder or condition is prevalent or more likely to occur.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

As used herein, the term “animal” includes, but is not limited to, humans and non-human vertebrates such as wild animals, rodents, such as rats, ferrets, and domesticated animals, and farm animals, such as dogs, cats, horses, pigs, cows, sheep, and goats. In some embodiments, the animal is a mammal. In some embodiments, the animal is a human. In some embodiments, the animal is a non-human mammal.

The term “vaccine” as used herein is meant to refer to a composition capable of generating immunity for the prophylaxis and/or treatment of diseases (e.g., viral infections). In some embodiments, the vaccine is a composition capable of generating therapeutically effective immunity for the prophylaxis or treatment of lentivirus infection or lentivirus replication or propagation in a subject. Accordingly, vaccines are medicaments which comprise antigens in protein and/or nucleic acid forms of those antigens and are in animals for generating specific defense and protective substance by vaccination. A “vaccine composition” can include a pharmaceutically acceptable excipient, carrier or diluent. A “vaccine composition” or “nucleic acid vaccine composition” as used herein can comprise a DNA vaccine, a RNA vaccine or a combination thereof. In some embodiments, vaccines of the disclosure comprise one or a plurality of aptamer disclosed herein or variants thereof that comprise at least about 70% sequence identity to the one or plurality of aptamers.

In some embodiments, the disclosure relates to a pharmaceutical composition comprising a therapeutically effective amount of an aptamer that is specific for an immunodominant epitope of a viral antigen and a therapeutically effective amount of an antigen comprising an immunotherapeutic epitope of the viral antigen. In some embodiments, the disclosure relates to a pharmaceutical composition comprising a therapeutically effective amount of an aptamer that is specific for an immunodominant epitope of a cancer antigen and a therapeutically effective amount of an antigen comprising an immunotherapeutic epitope of the cancer antigen.

Examples of vaccines that comprise HIV or SARS-COV2 or influenza antigens are known. In some embodiments, the spike protein or functional fragment thereof are provided or used as one or more protein antigenic sequences or as provided in nucleic acid sequences encoding one or more proteins comprising antigens. SARS-COV2 antigen sequences are known and have been administered in vaccines. Vaccines and methods of administering vaccines to subjects are described, in respect to SARS-COV2, in CN111533800, WO/2020/075955, WO/2022/018128, WO/2021/226405, WO/2022/020810, WO/2021/236415, WO/2022/061264, WO/2021/247412, WO/2021/231963, WO/2022/098728, and WO/2022/234416. The antigen sequences comprising epitopes as well as methods of administering those antigens are provided herein, and the above-identified references are incorporated by reference in their entireties. Spike protein of SARS-CoV2 is encoded by the following nucleic acid sequence:

| SEQ ID NO: 1. | |
|---|---|
| 1 | atgtttgttt ttcttgtttt attgccacta gtctctagtc agtgtgttaa tcttacaacc |
| 61 | agaactcaat taccccctgc atacactaat tctttcacac gtggtgttta ttaccctgac |
| 121 | aaagttttca gatcctcagt tttacattca actcaggact tgttcttacc tttcttttcc |
| 181 | aatgttactt ggttccatgc tatacatgtc tctgggacca atggtactaa gaggtttgat |
| 241 | aaccctgtcc taccatttaa tgatggtgtt tattttgctt ccactgagaa gtctaacata |
| 301 | ataagaggct ggatttttgg tactacttta gattcgaaga cccagtccct acttattgtt |
| 361 | aataacgcta ctaatgttgt tattaaagtc tgtgaatttc aattttgtaa tgatccattt |
| 421 | ttgggtgttt attaccacaa aaacaacaaa agttggatgg aaagtgagtt cagagtttat |
| 481 | tctagtgcga ataattgcac ttttgaatat gtctctcagc cttttcttat ggaccttgaa |
| 541 | ggaaaacagg gtaatttcaa aaatcttagg gaatttgtgt ttaagaatat tgatggttat |
| 601 | tttaaaatat attctaagca cacgcctatt aatttagtgc gtgatctccc tcagggtttt |
| 661 | tcggctttag aaccattggt agatttgcca ataggtatta acatcactag gtttcaaact |
| 721 | ttacttgctt tacatagaag ttatttgact cctggtgatt cttcttcagg ttggacagct |
| 781 | ggtgctgcag cttattatgt gggttatctt caacctagga cttttctatt aaaatataat |
| 841 | gaaaatggaa ccattacaga tgctgtagac tgtgcacttg accctctctc agaaacaaag |
| 901 | tgtacgttga aatccttcac tgtagaaaaa ggaatctatc aaacttctaa ctttagagtc |
| 961 | caaccaacag aatctattgt tagatttcct aatattacaa acttgtgccc ttttggtgaa |
| 1021 | gtttttaacg ccaccagatt tgcatctgtt tatgcttgga acaggaagag aatcagcaac |
| 1081 | tgtgttgctg attattctgt cctatataat tccgcatcat tttccacttt taagtgttat |
| 1141 | ggagtgtctc ctactaaatt aaatgatctc tgctttacta atgtctatgc agattcattt |
| 1201 | gtaattagag gtgatgaagt cagacaaatc gctccagggc aaactggaaa gattgctgat |
| 1261 | tataattata aattaccaga tgattttaca ggctgcgtta tagcttggaa ttctaacaat |
| 1321 | cttgattcta aggttggtgg taattataat tacctgtata gattgtttag gaagtctaat |
| 1381 | ctcaaacctt ttgagagaga tatttcaact gaaatctatc aggccggtag cacaccttgt |
| 1441 | aatggtgttg aaggttttaa ttgttacttt cctttacaat catatggttt ccaacccact |
| 1501 | aatggtgttg gttaccaacc atacagagta gtagtacttt cttttgaact tctacatgca |
| 1561 | ccagcaactg tttgtggacc taaaaagtct actaatttgg ttaaaaacaa atgtgtcaat |
| 1621 | ttcaacttca atggtttaac aggcacaggt gttcttactg agtctaacaa aaagtttctg |
| 1681 | cctttccaac aatttggcag agacattgct gacactactg atgctgtccg tgatccacag |
| 1741 | acacttgaga ttcttgacat tacaccatgt tcttttggtg gtgtcagtgt tataacacca |
| 1801 | ggaacaaata cttctaacca ggttgctgtt ctttatcagg atgttaactg cacagaagtc |
| 1861 | cctgttgcta ttcatgcaga tcaacttact cctacttggc gtgtttattc tacaggttct |
| 1921 | aatgtttttc aaacacgtgc aggctgttta ataggggctg aacatgtcaa caactcatat |
| 1981 | gagtgtgaca tacccattgg tgcaggtata tgcgctagtt atcagactca gactaattct |
| 2041 | cctcggcggg cacgtagtgt agctagtcaa tccatcattg cctacactat gtcacttggt |
| 2101 | gcagaaaatt cagttgctta ctctaataac tctattgcca tacccacaaa ttttactatt |
| 2161 | agtgttacca cagaaattct accagtgtct atgaccaaga catcagtaga ttgtacaatg |
| 2221 | tacatttgtg gtgattcaac tgaatgcagc aatcttttgt tgcaatatgg cagtttttgt |
| 2281 | acacaattaa accgtgcttt aactggaata gctgttgaac aagacaaaaa cacccaagaa |
| 2341 | gtttttgcac aagtcaaaca aatttacaaa acaccaccaa ttaaagattt tggtggtttt |
| 2401 | aatttttcac aaatattacc agatccatca aaaccaagca agaggtcatt tattgaagat |
| 2461 | ctacttttca acaaagtgac acttgcagat gctggcttca tcaaacaata tggtgattgc |
| 2521 | cttggtgata ttgctgctag agacctcatt tgtgcacaaa agtttaacgg ccttactgtt |
| 2581 | ttgccacctt tgctcacaga tgaaatgatt gctcaataca cttctgcact gttagcgggt |
| 2641 | acaatcactt ctggttggac ctttggtgca ggtgctgcat tacaaatacc atttgctatg |
| 2701 | caaatggctt ataggtttaa tggtattgga gttacacaga atgttctcta tgagaaccaa |
| 2761 | aaattgattg ccaaccaatt taatagtgct attggcaaaa ttcaagactc actttcttcc |
| 2821 | acagcaagtg cacttggaaa acttcaagat gtggtcaacc aaaatgcaca agctttaaac |
| 2881 | acgcttgtta aacaacttag ctccaatttt ggtgcaattt caagtgtttt aaatgatatc |
| 2941 | ctttcacgtc ttgacaaagt tgaggctgaa gtgcaaattg ataggttgat cacaggcaga |
| 3001 | cttcaaagtt tgcagacata tgtgactcaa caattaatta gagctgcaga aatcagagct |
| 3061 | tctgctaatc ttgctgctac taaaatgtca gagtgtgtac ttggacaatc aaaaagagtt |
| 3121 | gatttttgtg gaaagggcta tcatcttatg tccttccctc agtcagcacc tcatggtgta |
| 3181 | gtcttcttgc atgtgactta tgtccctgca caagaaaaga acttcacaac tgctcctgcc |
| 3241 | atttgtcatg atggaaaagc acactttcct cgtgaaggtg tctttgtttc aaatggcaca |
| 3301 | cactggtttg taacacaaag gaatttttat gaaccacaaa tcattactac agacaacaca |
| 3361 | tttgtgtctg gtaactgtga tgttgtaata ggaattgtca acaacacagt ttatgatcct |
| 3421 | ttgcaacctg aattagactc attcaaggag gagttagata aatattttaa gaatcataca |
| 3481 | tcaccagatg ttgatttagg tgacatctct ggcattaatg cttcagttgt aaacattcaa |
| 3541 | aaagaaattg accgcctcaa tgaggttgcc aagaatttaa atgaatctct catcgatctc |
| 3601 | caagaacttg gaaagtatga gcagtatata aaatggccat ggtacatttg gctaggtttt |
| 3661 | atagctggct tgattgccat agtaatggtg acaattatgc tttgctgtat gaccagttgc |
| 3721 | tgtagttgtc tcaagggctg ttgttcttgt ggatcctgct gcaaatttga tgaagacgac |
| 3781 | tctgagccag tgctcaaagg agtcaaatta cattacacat aa |
| Severe acute respiratory syndrome coronavirus 2 (SARS-COV-2) spike protein S (SEQ ID NO: 2): | |
| 1 | mfvflvllpl vssqcvnltt rtqlppaytn sftrgvyypd kvfrssvlhs tqdlflpffs |
| 61 | nvtwfhaihv sgtngtkrfd npvlpfndgv yfasteksni irgwifgttl dsktqslliv |
| 121 | nnatnvvikv cefqfondpf lgvyyhknnk swmesefrvy ssannctfey vsqpflmdle |
| 181 | gkqgnfknlr efvfknidgy fkiyskhtpi nlvrdlpqgf saleplvdlp iginitrfqt |
| 241 | llalhrsylt pgdsssgwta gaaayyvgyl qprtfllkyn engtitdavd caldplsetk |
| 301 | ctlksftvek giyqtsnfrv qptesivrfp nitnlcpfge vfnatrfasv yawnrkrisn |
| 361 | cvadysvlyn sasfstfkcy gvsptklndl cftnvyadsf virgdevrqi apgqtgkiad |
| 421 | ynyklpddft gcviawnsnn ldskvggnyn ylyrlfrksn lkpferdist eiyqagstpc |
| 481 | ngvegfncyf plqsygfqpt ngvgyqpyrv vvlsfellha patvcgpkks tnlvknkcvn |
| 541 | fnfngltgtg vltesnkkfl pfqqfgrdia dttdavrdpq tleilditpc sfggvsvitp |
| 601 | gtntsnqvav lyqdvnctev pvaihadqlt ptwrvystgs nvfqtragcl igaehvnnsy |
| 661 | ecdipigagi casyqtqtns prrarsvasq siiaytmslg aensvaysnn siaiptnfti |
| 721 | svtteilpvs mtktsvdctm yicgdstecs nlllqygsfc tqlnraltgi aveqdkntqe |
| 781 | vfaqvkqiyk tppikdfggf nfsqilpdps kpskrsfied llfnkvtlad agfikqygdc |
| 841 | lgdiaardli caqkingltv lpplltdemi aqytsallag titsgwtfga gaalqipfam |
| 901 | qmayrfngig vtqnvlyenq klianqfnsa igkiqdslss tasalgklqd vvnqnagaln |
| 961 | tlvkqlssnf gaissvlndi lsrldkveae vqidrlitgr lqslqtyvtq qliraaeira |
| 1021 | sanlaatkms ecvlgqskrv dfcgkgyhlm sfpqsaphgv vflhvtyvpa qeknfttapa |
| 1081 | ichdgkahfp regvfvsngt hwfvtqrnfy epqiittdnt fvsgncdvvi givnntvydp |
| 1141 | lqpeldsfke eldkyfknht spdvdlgdis ginasvvniq keidrlneva knlneslidl |
| 1201 | qelgkyeqyi kwpwyiwlgf iagliaivmv timlccmtsc csclkgcesc gscckfdedd |
| 1261 | sepvlkgvkl hyt |

In some embodiments, the antigen that comprises an epitope is a protein encoded by SEQ ID NO: 1 or a functional fragment thereof. In some embodiments, the immunodominant epitope is from about 8 to about 20 amino acids in length as a fragment of protein encoded by SEQ ID NO: 1. In some embodiments, the immunodominant epitope is from about 10 to about 20 amino acids in length as a fragment of protein encoded by SEQ ID NO: 1. In some embodiments, the immunodominant epitope is from about 10 to about 20 amino acids of SEQ ID NO: 2. In some embodiments, the antigen that comprises an epitope that is a protein encoded by SEQ ID NO: 1 or a functional fragment thereof that comprises at least 70%, 75%, 80%, 85%, 90%, 91%, 92%, 93%, 94%, 95%, 96%, 97%, 98% or 99% sequence identity to SEQ ID NO:1. In some embodiments, the immunodominant epitope is from about 10 to about 20 amino acids in length as a fragment of protein chosen from SEQ ID NO: 1, or a functional fragment thereof that that comprises at least 70%, 75%, 80%, 85%, 90%, 91%, 92%, 93%, 94%, 95%, 96%, 97%, 98% or 99% sequence identity from about 10 to about 20 amino acids encoded by SEQ ID NO: 1. In some embodiments, the immunodominant epitope is from about 10 to about 20 amino acids of SEQ ID NO: 2 or a functional fragment thereof that that comprises at least 70%, 75%, 80%, 85%, 90%, 91%, 92%, 93%, 94%, 95%, 96%, 97%, 98% or 99% sequence identity to SEQ ID NO: 2.

In some embodiments, the HIV Env protein or functional fragment thereof are provided or used as one or more protein antigenic sequences or as provided in nucleic acid sequences encoding one or more proteins comprising an HIV immunodominant epitopes and/or one or more proteins comprising an HIV immunotherapeutic epitope. HIV antigen sequences are known and have been administered in vaccines. Vaccines and methods of administering vaccines to subjects are described, in respect to HIV antigens in WO/2014/039840, WO/2020/046982, WO/2006/092046, WO/2010/040136, WO/2020/251389, WO/2019/089817, WO/2010/073291. The antigen sequences comprising epitopes as well as methods of administering those antigens are provided herein, and the above-identified references are incorporated by reference in their entireties. gp120 protein of HIV is encoded by the following mRNA sequence:

| SEQ ID NO: 3 | |
|---|---|
| | gttcctgtgt ggaaagatgc agagaccacc ttattttgtg catcagatgc caaagcacat |
| 61 | gagacagaag tgcacaatgt ctgggccaca catgcctgtg tacccacaga ccccaaccca |
| 121 | caagaaatac aactgaaaaa tgtaacagag aattttaaca tgtggaaaaa taacatggta |
| 181 | gagcagatgc aggaggatgt aatcagttta tgggatcaaa gtctaaagcc atgtgtaaag |
| 241 | ttaactcctc tctgcgttac tttaaattgt accgatgcta ctttgaccaa tagcacttac |
| 301 | ataaccaatg tctctaagat aataggagat ataacagagg aagtaagaaa ctgttctttt |
| 361 | aatatgacca cagaactaag agataagaag cagaaggtcc atgcactttt tttataagct |
| 421 | tgatatagta gaaattgaaa agaataggaa tgagtatagg ttaataaatt gtaatacttc |
| 481 | ggtcattaag caggcttgtc caaagatatc ctttgatcca attcctatac attattgtac |
| 541 | tccagctggt tatgcgattt taaagtgtaa tgataagaat ttcaatggga cagggccatg |
| 601 | taaaaatgtc agctcagtac aatgcacaca tggaattaag ccagtggtat caactcaatt |
| 661 | gctgttaaat ggcagtctag cagaagaaga gataataatc agatctgaaa atctcacaaa |
| 721 | caatgccaaa accataatag tgcaccttaa taaatctgta gaaatcaatt gtaccagacc |
| 781 | cttcaacaat acaagaacaa gtataactat aggaccagga caaatgttct atagaacagg |
| 841 | agagataata ggagatataa caaaagcata ttgtgagatt aatggaacaa aatggaatga |
| 901 | aactttaaaa caggtagctg aaaaactaaa agagcacttt aataataaga caatagtctt |
| 961 | tcaaccaccc tcaggaggag atctagaaat tacaatgcat cattttaatt gtagagggga |
| 1021 | atttttctat tgcaatacaa cacgactgtt taatcatact tacatggaaa ataaaaccat |
| 1081 | gggggggtgt aatgacacta tcatacttcc atgcaagata aagaaaatta taaatatgtg |
| 1141 | gcagggagta ggacaagcaa tgtatgctcc tcccatcagg ggaagcatta attgtgtatc |
| 1201 | aaatattaca ggaatactat tgataagaga tggtggtgat aataatgcga ctaacgagac |
| 1261 | cttcagacct ggaggaggaa atataaagga caattggaga agtgaattat ataaatataa |
| 1321 | agtagtacaa attgaaccac taggaatagc acccaccagg gcaaagggaa gagtggtgga |
| 1381 | gagagaaaga aga |
| HIV gp41 nucleic acid sequence (SEQ ID NO: 4) | |
| 1 | aacaacatat gttgcaactc acagtctggg gcataaagca gctccaggca agagtcctgg |
| 61 | ctgtggaaag atacctaaag gatcaacagc tcctggggat ttggggttgc tctggaaaac |
| 121 | tcatttgcac cactactgtg ccttggaatg ctagttggag taataaatct ctgaatgaca |
| 181 | tttggaataa catgacctgg atggagtggg aaagagaaat tggcaattac acaggcttaa |
| 241 | tatacacctt aattgaacaa tcacagaacc agcaagaaaa gaatgaacaa gaattattgg |
| 301 | aattggataa atgggcaagt ttgtggaatt ggtttgacat aacaaaatgg ttgtggtata |
| 361 | taaaaatatt ca |
| HIV gp41 protein sequence (SEQ ID NO: 5): | |
| | NLLRAIEAQQ HLLQLTVWGI KQLQARILAV ERYLQDQQLL GSWGCSGRHI |
| | CTTNVPWNASWSNKSLDEIW GNMTWIEWER EIDNYTGLIY NLIEESQTQQ |
| | EKNEQDLLQL DKWASLWNWFSITKWLWYI |
| HIV gp120 protein sequence (SEQ ID NO: 6): | |
| 1 | sateklwvtv yygvpvwkea tttlfcasda kaydtevhnv wathacvptd pnpqevvlvn |
| 61 | vtenfnmwkn dmveqmhedi islwdqslkp cvkltplcvs lkctdlkndt ntnsssgrmi |
| 121 | mekgeikncs fnistsirgk vqkeyaffyk ldiipidndt tsykltsent svitqacpkv |
| 181 | sfepipihyc apagfailkc nnktfngtgp ctnvstvqct hgirpvvstq lllngslaee |
| 241 | evvirsvnft dnaktiivql ntsveinctr pnnntrkrir iqrgpgrafv tigkignmrq |
| 301 | ahcnisrakw nntlkqiask lreqfgnnkt iifkqssggd peivthsfnc ggeffycnst |
| 361 | qlfnstwfns twstegsnnt egsdtitlpc rikqiinmwq kvgkamyapp isgqircssn |
| 421 | itgllltrdg gnsnneseif rpgggdmrdn wrselykykv vkieplgvap tkakrrvvqr |
| 481 | ekr |
| gp160 precursor protein sequence (SEQ ID NO: 7): | |
| 1 | mrvkekyqhl wrwgwrwgtm llgmlmlcsa teklwvtvyy gvpvwkeatt tlfcasdaka |
| 61 | ydtevhnvwa thacvptdpn pqevvlvnvt enfnmwkndm veqmhediis lwdqslkpcv |
| 121 | kltplcvslk ctdlkndtnt nsssgrmime kgeikncsfn istsirgkvq keyaffykld |
| 181 | iipidndtts ykltsentsv itqacpkvsf epipihycap agfailkcnn ktfngtgpct |
| 241 | nvstvqcthg irpvvstqll lngslaeeev virsvnftdn aktiivqlnt sveinctrpn |
| 301 | nntrkririq rgpgrafvti gkignmrqah cnisrakwnn tlkqiasklr eqfgnnktii |
| 361 | fkqssggdpe ivthsfncgg effyenstql fnstwfnstw stegsnnteg sdtitlpcri |
| 421 | kqiinmwqkv gkamyappis ggircssnit gllltrdggn snneseifrp gggdmrdnwr |
| 481 | selykykvvk ieplgvaptk akrrvvgrek ravgigalfl gflgaagstm gaasmtltvq |
| 541 | arqllsgivq qqnnllraie aqqhllqltv wgikqlqari laverylkdq qllgiwgcsg |
| 601 | klicttavpw naswsnksle qiwnhttwme wdreinnyts lihslieesq nqqekneqel |
| 661 | leldkwaslw nwfnitnwlw yiklfimivg glvglrivfa vlsivnrvrq gysplsfqth |
| 721 | lptprgpdrp egieeegger drdrsirlvn gslaliwddl rslclfsyhr lrdlllivtr |
| 781 | ivellgrrgw ealkywwnll qywsqelkns avsllnatai avaegtdrvi evvqgacrai |
| 841 | rhiprrirqg lerill |

Underlined and bold is an embodiment of gp120.

In some embodiments, the antigen that comprises an epitope that is a protein encoded by SEQ ID NO: 3 or 4 or a functional fragment thereof. In some embodiments, the immunodominant epitope is from about 10 to about 20 amino acids in length as a fragment of protein chosen from SEQ ID NO: 3 or 4, or a functional fragment thereof. In some embodiments, the immunodominant epitope is from about 10 to about 20 amino acids of SEQ ID NO: 5. In some embodiments, the immunodominant epitope is from about 10 to about 20 amino acids of SEQ ID NO: 6. In some embodiments, the antigen that comprises an epitope that is a protein encoded by SEQ ID NO: 3 or 4 or a functional fragment thereof that comprises at least 70%, 75%, 80%, 85%, 90%, 91%, 92%, 93%, 94%, 95%, 96%, 97%, 98% or 99% sequence identity to SEQ ID NO: 3 or SEQ ID NO: 4. In some embodiments, the immunodominant epitope is from about 10 to about 20 amino acids in length as a fragment of protein chosen from SEQ ID NO: 3 or 4, or a functional fragment thereof that that comprises at least 70%, 75%, 80%, 85%, 90%, 91%, 92%, 93%, 94%, 95%, 96%, 97%, 98% or 99% sequence identity from about 10 to about 20 amino acids encoded by SEQ ID NO: 3 or SEQ ID NO: 4. In some embodiments, the immunodominant epitope is from about 10 to about 20 amino acids of SEQ ID NO: 5 or a functional fragment thereof that that comprises at least 70%, 75%, 80%, 85%, 90%, 91%, 92%, 93%, 94%, 95%, 96%, 97%, 98% or 99% sequence identity to SEQ ID NO: 5. In some embodiments, the immunodominant epitope is from about 10 to about 20 amino acids of SEQ ID NO: 6, or a functional fragment thereof that that comprises at least 70%, 75%, 80%, 85%, 90%, 91%, 92%, 93%, 94%, 95%, 96%, 97%, 98% or 99% sequence identity to SEQ ID NO: 6. In some embodiments, the immunodominant epitope is from about 10 to about 20 amino acids of SEQ ID NO: 7 or a functional fragment thereof that that comprises at least 70%, 75%, 80%, 85%, 90%, 91%, 92%, 93%, 94%, 95%, 96%, 97%, 98% or 99% sequence identity to SEQ ID NO: 7.

Influenzas vaccines are known, including.
Influenza Virus Vaccine, Quadrivalent, Types A and Types B.
Influenza Virus Vaccine, Trivalent, Types A and B.
Influenza Virus Vaccine, H5N1 (for National Stockpile).

In some embodiments, the immunodominant epitopes are derived from HA antigen of influenza virus. In some embodiments, the immunodominant and/or immunotherapeutic epitopes are derived from sequences disclosed in WO2015050177, WO2012164928, WO2015093996, WO2001022992, all of which are incorporated by reference in their entireties. In some embodiments, the Influenza HA Type A protein is SEQ ID NO: 8

| SEQ ID NO: 8: | |
|---|---|
| 1 | mkakllillc tlsatdadti cigyhannst etvdtvlekn vtvthsvnll edshngklcr |
| 61 | lkgitplqlg kcsiagwilg npeceslfsk kswsyiaetp npengicypg yfsdyeelre |
| 121 | qlssvssfer feifpkessw pkhsinkgvt ascshkgksn fyrnllwlte kngsypnlsk |
| 181 | syvndkekev lvlwgvhhps niedqraiyr ketayvsvva shysrrftpe iarrpkirdq |
| 241 | egrinyywtl lgprdtiife angnliapwy afalsrgfks giiisnasmd dcdtkcqtpq |
| 301 | gainsslpfq svhpvtigec pkyvrstklr mvtglrnips iqsrglfgai agfieggwtg |
| 361 | midgwygyhh qneqgsgyaa dqkstqnain gitnkvnsvi ekmntqftav gkefnklekr |
| 421 | menlnkkvdd gfldiwtyna ellvllener tldfhdsnvk slyekvkgql knnakeigng |
| 481 | cfefyhkcdn ecmdsvkngt ydypkysees klnrekidgv elksmgvyqi laiystvass |
| 541 | lvllvslgai sfwmcsngsl qcrici |

In some embodiments, the Influenza HA Type A protein is Human HA Type A H7N9 (SEQ ID NO: 9).

| | |
|---|---|
| 1 | mntqilvfal iaiiptnadk iclghhavsn gtkvntlter gvevvnatet vertnipric |
| 61 | skgkrtvdlg qcgllgtitg ppqcdqflef sadliierre gsdvcypgkf vneealrqil |
| 121 | resggidkea mgftysgirt ngatsacrrs gssfyaemkw llsntddaaf pqmtksyknt |
| 181 | rkspalivwg ihhsvstaeq tklygsgnkl vtvgssnyqq sfvpspgarp qvnglsgrid |
| 241 | fhwlmlnpnd tvtfsfngaf iapdrasflr gksmgiqsgv qvdancegdc yhsggtiisn |
| 301 | lpfqnidsra vgkcpryvkq rslllatgmk nvpeipkgrg lfgaiagfie ngweglidgw |
| 361 | ygfrhqnaqg egtaadykst qsaidqitgk lnrliektnq qfelidnefn evekqignvi |
| 421 | nwtrdsitev wsynaellva menqhtidla dsemdklyer vkrqlrenae edgtgcfeif |
| 481 | hkcdddcmas irnntydhsk yreeamqnri qidpvklssg ykdvilwfsf gascfillai |
| 541 | vmglvficvk ngnmrctici |

In some embodiments, the immunodominant epitope is from about 10 to about 20 amino acids of SEQ ID NO: 8 or a functional fragment thereof that that comprises at least 70%, 75%, 80%, 85%, 90%, 91%, 92%, 93%, 94%, 95%, 96%, 97%, 98% or 99% sequence identity to SEQ ID NO: 8. In some embodiments, the immunodominant epitope is from about 10 to about 20 amino acids of SEQ ID NO: 9 or a functional fragment thereof that that comprises at least 70%, 75%, 80%, 85%, 90%, 91%, 92%, 93%, 94%, 95%, 96%, 97%, 98% or 99% sequence identity to SEQ ID NO: 9.

“Variants” are intended to mean substantially similar sequences. For nucleic acid molecules, a variant comprises a nucleic acid molecule having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. In some embodiments, a variant is a functional fragment if a sequence identifier disclosed herein. As used herein, a “native” nucleic acid molecule or polypeptide comprises a naturally occurring or endogenous nucleotide sequence or amino acid sequence, respectively. For nucleic acid molecules, conservative variants include those sequences that, because of the degeneracy of the genetic code, encode the amino acid sequence of one of the polypeptides of the disclosure. Variant nucleic acid molecules also include synthetically derived nucleic acid molecules, such as those generated, for example, by using site-directed mutagenesis but which still encode a protein of the disclosure. Generally, variants of a particular nucleic acid molecule of the disclosure will have at least about 70%, 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein. Variants of a particular nucleic acid molecule of the disclosure (i.e., the reference DNA sequence) can also be evaluated by comparison of the percent sequence identity between the polypeptide encoded by a variant nucleic acid molecule and the polypeptide encoded by the reference nucleic acid molecule. Percent sequence identity between any two polypeptides can be calculated using sequence alignment programs and parameters described elsewhere herein. Where any given pair of nucleic acid molecule of the disclosure is evaluated by comparison of the percent sequence identity shared by the two polypeptides that they encode, the percent sequence identity between the two encoded polypeptides is at least about 70%, 75%, 80%, 85%, 90%, 91%, 92%, 93%, 94%, 95%, 96%, 97%, 98%, 99% or more sequence identity. In some embodiments, the term “variant” protein is intended to mean a protein derived from the native protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native or wild-type protein upon which the variant structure is based. Variant proteins encompassed by the present disclosure are biologically active, that is they continue to possess the desired biological activity of the native protein as described herein. Such variants may result from, for example, genetic polymorphism or from human manipulation. Biologically active variants of a protein of the disclosure will have at least about 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the disclosure may differ from that protein by as few as from about 1 to about 15 amino acid residues, as few as from about 1 to about 10, or from about 6 to about 10, as few as about 5, as few as about 4, about 3, about 2, or about 1 amino acid residue. The proteins or polypeptides of the disclosure may be altered in various ways including amino acid substitutions, deletions, truncations, and insertions. Methods for such manipulations are generally known in the art. For example, amino acid sequence variants and fragments of the proteins can be prepared by mutations in the nucleic acid sequence that encode the amino acid sequence recombinantly. In some embodiments, the nucleic acid molecules or the nucleic acid sequences comprise conservative mutations of about 1, 2, 3, 4, 5, 6, 7, 8, 9, 10 or more nucleotides of the expressible nucleic acid sequence.

The “percent identity” or “percent homology” of two polynucleotide or two polypeptide sequences is determined by comparing the sequences using the GAP computer program (a part of the GCG Wisconsin Package, version 10.3 (Accelrys, San Diego, Calif.)) using its default parameters. “Identical” or “identity” as used herein in the context of two or more nucleic acids or amino acid sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may he performed manually or by using a computer sequence algorithm such as BLAST or BLAST 2.0. Briefly, the BLAST algorithm, which stands for Basic Local Alignment Search Tool is suitable for determining sequence similarity. Software for performing BLAST analyses is publicly available through the National Center for Biotechnology Information (http://www.ncbi.nlm.nih.gov). This algorithm involves first identifying high scoring sequence pair (HSPs) by identifying short words of length Win the query sequence that either match or satisfy some positive-valued threshold score T when aligned with a word of the same length in a database sequence. T is referred to as the neighborhood word score threshold (Altschul et al., supra). These initial neighborhood word hits act as seeds for initiating searches to find HSPs containing them. The word hits are extended in both directions along each sequence for as far as the cumulative alignment score can be increased. Extension for the word hits in each direction are halted when: 1) the cumulative alignment score falls off by the quantity X from its maximum achieved value; 2) the cumulative score goes to zero or below, due to the accumulation of one or more negative-scoring residue alignments; or 3) the end of either sequence is reached. The Blast algorithm parameters W, T and X determine the sensitivity and speed of the alignment. The Blast program uses as defaults a word length (W) of 11, the BLOSUM62 scoring matrix (see Henikoff et al., Proc. Natl. Acad. Sci. USA, 1992, 89, 10915-10919, which is incorporated herein by reference in its entirety) alignments (B) of 50, expectation (E) of 10, M=5, N=4, and a comparison of both strands. The BLAST algorithm (Karlin et al., Proc. Natl. Acad. Sci. USA, 1993, 90, 5873-5787, which is incorporated herein by reference in its entirety) and Gapped BLAST perform a statistical analysis of the similarity between two sequences. One measure of similarity provided by the BLAST algorithm is the smallest sum probability (P(N)), which provides an indication of the probability by which a match between two nucleotide sequences or amino acid sequences would occur by chance. For example, a nucleic acid is considered similar to another if the smallest sum probability in comparison of the test nucleic acid to the other nucleic acid is less than about 1, less than about 0.1, less than about 0.01, and less than about 0.001. Two single-stranded polynucleotides are “the complement” of each other if their sequences can be aligned in an anti-parallel orientation such that every nucleotide in one polynucleotide is opposite its complementary nucleotide in the other polynucleotide, without the introduction of gaps, and without unpaired nucleotides at the 5′ or the 3′ end of either sequence. A polynucleotide is “complementary” to another polynucleotide if the two polynucleotides can hybridize to one another under moderately stringent conditions. Thus, a polynucleotide or the nucleic acid sequence can be complementary to another polynucleotide without being its complement.

### Compositions

Described herein are compositions (including pharmaceutical compositions) and methods for the design, preparation, manufacture and/or formulation of aptamers where at least one region of the aptamer binds to or associates with an immunodominant epitope of an antigen. In some embodiments, the antigen is a viral antigen or a cancer antigen. As such the present invention is directed, in part, to polynucleotides encoding aptamers and/or antigens or polypeptides that are antigens comprising immunodominant epitopes. In some embodiments, the compositions of the disclosure relate to specifically polynucleotides, chimeric polynucleotides and/or circular polynucleotides encoding one or more immunotherapeutic epitopes or targeted adaptive vaccines or components thereof.

The disclosure relates to a composition comprising an aptamer specific for, or that specifically binds to, an immunodominant epitope of an antigen. In some embodiments, the antigen comprises an immunotherapeutic epitope and an immunodominant epitope. In some embodiments, the antigen comprises a viral antigen. In some embodiments, the antigen comprises an HIV-1 antigen, a SARS-COV2 antigen and/or an influenza antigen. In some embodiments, the antigen comprises one or a plurality of immunodominant epitopes and one or a plurality of immunotherapeutic epitopes.

Embodiments of the disclosure include compositions comprising aptamers that can bind or are specific for one or a plurality of immunodominant epitopes of Table Y.

TABLE Y
Immunodominant Epitopes separated by Antigen-type

| | |
|---|---|
| SARS-CoV2 epitopes | |
| Epitopes comprising | Relative to |
| AA residues 440- | SEQ ID NO: 2 |
| 460 | |
| Epitope comprising | |
| AA residue 369 | |
| HIV-1 epitopes | |
| The base of | Relative to |
| gp41N355/N289 | SEQ ID NO: 5 |
| Glycan holes at AA | Relative to |
| 230, 241, 289 and 344. | SEQ ID NO: 6 |
| V3 crown including | |
| AA residues 306, 308 | |
| and 316 | |
| Influenza epitopes | |
| 103-RESGG-107 in | Relative to |
| the HA globular | SEQ ID NO: 9 |
| head | |
| 373-TAA-375 in the | |
| stalk | |
| 220Q-225S-227G | |
| 431W-433Y-437L | |
| 351N-356L-361Y- | |
| 362G | |

Aptamer of the disclosure can be of varying length or composition depending upon the binding properties of the aptamer to the immunodominant epitope. In some embodiments, the aptamers are from about 10 to about 50 nucleotides in length. In some embodiments, the aptamers are from about 15 to about 50 nucleotides in length. In some embodiments, the aptamers are from about 20 to about 50 nucleotides in length. In some embodiments, the aptamers are from about 25 to about 50 nucleotides in length. In some embodiments, the aptamers are from about 30 to about 50 nucleotides in length. In some embodiments, the aptamers are from about 35 to about 50 nucleotides in length. In some embodiments, the aptamers are from about 40 to about 50 nucleotides in length. In some embodiments, the aptamers are from about 45 to about 50 nucleotides in length. In some embodiments, the aptamers are from about 10 to about 45 nucleotides in length. In some embodiments, the aptamers are from about 10 to about 40 nucleotides in length. In some embodiments, the aptamers are from about 10 to about 35 nucleotides in length. In some embodiments, the aptamers are from about 10 to about 30 nucleotides in length. In some embodiments, the aptamers are from about 10 to about 25 nucleotides in length. In some embodiments, the aptamers are from about 10 to about 20 nucleotides in length. In some embodiments, the aptamers are from about 15 to about 50 nucleotides in length. In some embodiments, the aptamers are from about 15 to about 45 nucleotides in length. In some embodiments, the aptamers are from about 15 to about 40 nucleotides in length. In some embodiments, the aptamers are from about 15 to about 35 nucleotides in length. In some embodiments, the aptamers are from about 10 to about 100 nucleotides in length.

Aptamers of the disclosure may have various ranges of equilibrium disassociation constants depending upon the nature of the aptamer and its epitope target. In some embodiments, the aptamer comprises a K_{D} value from about 10 to about 800 nM relative to the immunodominant epitope target to which it binds. In some embodiments, the aptamer comprises a K_{D} value from about 10 to about 800 nM relative to the immunodominant epitope target to which it binds. In some embodiments, the aptamer comprises a K_{D} value from about 50 to about 800 nM relative to the immunodominant epitope target to which it binds. In some embodiments, the aptamer comprises a K_{D} value from about 10 to about 700 nM relative to the immunodominant epitope target to which it binds. In some embodiments, the aptamer comprises a K_{D} value from about 10 to about 600 nM relative to the immunodominant epitope target to which it binds. In some embodiments, the aptamer comprises a K_{D} value from about 10 to about 500 nM relative to the immunodominant epitope target to which it binds. In some embodiments, the aptamer comprises a K_{D} value from about 10 to about 400 nM relative to the immunodominant epitope target to which it binds. In some embodiments, the aptamer comprises a K_{D} value from about 10 to about 300 nM relative to the immunodominant epitope target to which it binds. In some embodiments, the aptamer comprises a K_{D} value from about 10 to about 200 nM relative to the immunodominant epitope target to which it binds. In some embodiments, the aptamer comprises a K_{D} value from about 50 to about 200 nM relative to the immunodominant epitope target to which it binds. In some embodiments, the aptamer comprises a K_{D} value from about 1 nM to about 250 nM relative to the immunodominant epitope target to which it binds. In some embodiments, the aptamer comprises a K_{D} value from about 1 nM to about 800 nM relative to the immunodominant epitope target to which it binds. In some embodiments, the aptamer comprises a K_{D} value from about 1 nM to about 500 nM relative to the immunodominant epitope target to which it binds. In some embodiments, the aptamer comprises a K_{D} value from about 1 nM to about 400 nM relative to the immunodominant epitope target to which it binds. In some embodiments, the aptamer comprises a K_{D} value from about 1 nM to about 250 nM relative to the immunodominant epitope target to which it binds. In some embodiments, the aptamer comprises a K_{D} value from about 1 nM to about 150 nM relative to the immunodominant epitope target to which it binds.

In some embodiments, the aptamer comprises a K_{D} value of no more than about 800 nM relative to the immunodominant epitope target to which it binds. In some embodiments, the aptamer comprises a K_{D} value of no more than about 700 nM relative to the immunodominant epitope target to which it binds. In some embodiments, the aptamer comprises a K_{D} value of no more than about 600 nM relative to the immunodominant epitope target to which it binds. In some embodiments, the aptamer comprises a K_{D} value of no more than about 500 nM relative to the immunodominant epitope target to which it binds. In some embodiments, the aptamer comprises a K_{D} value of no more than about 400 nM relative to the immunodominant epitope target to which it binds. In some embodiments, the aptamer comprises a K_{D} value of no more than about 300 nM relative to the immunodominant epitope target to which it binds. In some embodiments, the aptamer comprises a K_{D} value of no more than about 200 nM relative to the immunodominant epitope target to which it binds.

In some embodiments, the aptamer comprises a K_{D} value of no more than about 450 nM relative to the immunodominant epitope target to which it binds. In some embodiments, the aptamer comprises a K_{D} value of no more than about 350 nM relative to the immunodominant epitope target to which it binds. In some embodiments, the aptamer comprises a K_{D} value of no more than about 250 nM relative to the immunodominant epitope target to which it binds. In some embodiments, the aptamer comprises a K_{D} value of no more than about 150 nM relative to the immunodominant epitope target to which it binds. In some embodiments, the aptamer comprises a K_{D} value of no more than about 50 nM relative to the immunodominant epitope target to which it binds.

In some embodiments, the aptamer comprises a K_{D} value of no more about 750 nM relative to the immunodominant epitope target to which it binds. In some embodiments, the aptamer comprises a K_{D} value of no more about 800 nM relative to the immunodominant epitope target to which it binds. In some embodiments, the aptamer comprises a K_{D} value of no more about 850 nM relative to the immunodominant epitope target to which it binds. In some embodiments, the aptamer comprises a K_{D} value of no more about 900 nM relative to the immunodominant epitope target to which it binds. In some embodiments, the aptamer comprises a K_{D} value of no more about 950 nM relative to the immunodominant epitope target to which it binds. In some embodiments, the aptamer comprises a K_{D} value of no more than about 1 micromolar relative to the immunodominant epitope target to which it binds. In some embodiments, the aptamer comprises a K_{D} value of no more than about 1.1 μM relative to the immunodominant epitope target to which it binds. In some embodiments, the aptamer comprises a K_{D} value of no more than about 1.2 μM relative to the immunodominant epitope target to which it binds. In some embodiments, the aptamer comprises a K_{D} value of no more than about 1.3 μM relative to the immunodominant epitope target to which it binds. In some embodiments, the aptamer comprises a K_{D} value of no more than about 1.4 μM relative to the immunodominant epitope target to which it binds.\

In some embodiments, the aptamer comprises a K_{D} value of no more about 750 nM relative to the immunodominant epitope target to which it binds. In some embodiments, the aptamer comprises a K_{D} value of no more about 800 nM relative to the immunodominant epitope target to which it binds. In some embodiments, the aptamer comprises a K_{D} value of no more about 850 nM relative to the immunodominant epitope target to which it binds. In some embodiments, the aptamer comprises a K_{D} value of no more about 900 nM relative to the immunodominant epitope target to which it binds. In some embodiments, the aptamer comprises a K_{D} value of no more about 950 nM relative to the immunodominant epitope target to which it binds. In some embodiments, the aptamer comprises a K_{D} value of no more than about 1 micromolar relative to the immunodominant epitope target to which it binds. In some embodiments, the aptamer comprises a K_{D} value of no more than about 1.1 μM relative to the immunodominant epitope target to which it binds. In some embodiments, the aptamer comprises a K_{D} value of no more than about 1.2 μM relative to the immunodominant epitope target to which it binds. In some embodiments, the aptamer comprises a K_{D} value of no more than about 1.3 μM relative to the immunodominant epitope target to which it binds. In some embodiments, the aptamer comprises a K_{D} value of no more than about 1.4 μM relative to the immunodominant epitope target to which it binds.

Embodiments of the disclosure include a composition comprising one or a plurality of aptamers, the aptamer being present on a plasmid. A plasmid may comprise a nucleic acid sequence that encodes one or more of the various immunotherapeutic epitopes disclosed herein including coding sequences that encode synthetic, consensus antigen capable of eliciting an immune response against one or a plurality of viral proteins.

A single plasmid may contain coding sequence for a single viral immunogen or epitops, coding sequence for two viral immunogens or epitopes, coding sequence for three filoprotein immunogens, coding sequence for four viral immunogens or epitopes, coding sequence for five viral immunogens or epitopes; or coding sequences for six viral immunogens or epitopes. A single plasmid may contain a coding sequence for a single viral immunogen which can be formulated together. In some embodiments, a plasmid may comprise coding sequence that encodes IL-12, IL-15 and/or IL-28.

The plasmid may further comprise an initiation codon, which may be upstream of the coding sequence, and a stop codon, which may be downstream of the coding sequence. The initiation and termination codon may be in frame with the coding sequence that comprises the aptamer or a reverse complementary sequence of the aptamer.

The plasmid may also comprise a promoter that is operably linked to the coding sequence comprising the aptamer. The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human actin, human myosin, human hemoglobin, human muscle creatine, or human metalothionein. The promoter may also be a tissue specific promoter, such as a muscle or skin specific promoter, natural or synthetic. Examples of such promoters are described in US patent application publication no. US20040175727, the contents of which are incorporated herein in its entirety.

The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen, San Diego, CA).

The plasmid may also comprise an enhancer upstream of the coding sequence. The enhancer may be human actin, human myosin, human hemoglobin, human muscle creatine or a viral enhancer such as one from CMV, FMDV, RSV or EBV. Polynucleotide function enhances are described in U.S. Pat. Nos. 5,593,972, 5,962,428, and WO94/016737, the contents of each are fully incorporated by reference.

The plasmid may also comprise a mammalian origin of replication in order to maintain the plasmid extrachromosomally and produce multiple copies of the plasmid in a cell. The plasmid may be pVAX1, pCEP4 or pREP4 from Invitrogen (San Diego, CA), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid.

The plasmid may also comprise a regulatory sequence, which may be well suited for gene expression in a cell into which the plasmid is administered. The coding sequence may comprise one or a plurality of codons that may allow more efficient transcription of the coding sequence in the host cell.

The coding sequence may also comprise an Ig leader sequence. The leader sequence may be 5′ of the aptamer sequence. The aptamers encoded by this sequence may comprise an N-terminal Ig leader followed by a consensus aptamer sequence. The N-terminal Ig leader may be IgE or IgG. In some embodiments, the coding sequence comprises a first and second nucleotide sequence, the first nucleic acid sequence is an aptamer disclosed herein and the second nucleic acid encoding one or a plurality of antigens comprising one or a plurality of epitopes. In some embodiments, a composition comprising one or a plurality of aptamers disclosed herein and a plasmid comprising a coding sequence that comprises at least a first nucleic acid sequence encoding a first immunotherapeutic epitope. In some embodiments, a composition comprising one or a plurality of aptamers disclosed herein and a plasmid comprising a coding sequence that comprises at least a first nucleic acid sequence encoding a first immunotherapeutic epitope and one or more immunodominant epitopes. m

The plasmid may be pSE420 (Invitrogen, San Diego, Calif), which may be used for protein production in Escherichia coli (E. coli). The plasmid may also be pYES2 (Invitrogen, San Diego, Calif), which may be used for protein production in Saccharomyces cerevisiae strains of yeast. The plasmid may also be of the MAXBAC™ complete baculovirus expression system (Invitrogen, San Diego, Calif), which may be used for protein production in insect cells. The plasmid may also be pcDNA I or pcDNA3 (Invitrogen, San Diego, Calif), which may be used for protein production in mammalian cells such as Chinese hamster ovary (CHO) cells. 5.

### Compositions

Compositions are provided which comprise nucleic acid molecules. The compositions may comprise a plurality of copies of a single nucleic acid molecule such a single plasmid, a plurality of copies of two or more different nucleic acid molecules such as two or more different plasmids. For example, a composition may comprise plurality of two, three, four, five, six, seven, eight, nine or ten or more different nucleic acid molecules. Such compositions may comprise plurality of two, three, four, five, six, or more different plasmids.

TABLE Z
Non-Limiting Examples of Immunotherapeutic
Epitopes separated by Antigen-type

| SARS-CoV2 epitopes | |
|---|---|
| RBD C1 | Liu and Wilson, |
| RBD C1D | Immunological |
| RBD C2 | Reviews, 2022 |
| RBD C3 | |
| RBD C4 | |
| HIV-1 epitopes | West et al, Cell |
| V1V2 epitope | 2014; Burton |
| V3-glycan epitope | and Hangartner, |
| CD4 binding site epitope | Annu. Rev. |
| Gp120-gp41 interface | |
| Silent face epitope | |
| C3/V5 epitope | |
| Fusion peptide epitope | |
| Influenza epitopes | |
| With numbering relative | |
| to SEQ ID NO: 9 below | |

| Contacts on HA1 | Contacts on HA2 |
|---|---|
| H5: H44, Q46, | V364, D365, G366, W367, |
| D47, I48, S304, | A382, K384, T387, Q388, I391, |
| M305, P306 | D392, V394, T395, V398, I402 |
| H7: N53, T55, | I366, D367, G368, W369, A384, |
| E56, T57, N307, | Y386, T389, Q390, I393, D394, |
| L308, P309 | I396, T397, L400, I404 |
| H3: N54, T56, | I363, D364, G365, W366, A381, |
| E57, L58, D307, | L383, T386, Q387, I390, D391, |
| K308, P309 | I393, N394, L397, I401 |
| H1: H45, S46, | V362, D363, G364, W365, |
| S306, T333 | L382, K385, T386, Q387, N388, |
| | I389, D390, T393, N397, I400, |
| | E401 |
| H24, H44, Q46, | V364, D365, G366, W367, |
| S304, M305 | K384, T387, Q388, I391, T395, |
| | V398, N399, I402 |
| H25, H45, V46, | D363, G364, W365, Q382, |
| N47, L496, S306, | T385, Q386, I389, D390, T393, |
| L307, P308, T333 | V396, N397, I400 |
| H3: Y115, T148, | |
| G151, G152, S153, | |
| S154, W170, | |
| Y172, K173, S174, | |
| G175, S176, K206, | |
| E207, N210, L211, | |
| V213, L243 | |
| H2: H43, K45, I47, | V358, D359, G360, W361, |
| T302, L303, P304, | K378, T381, Q382, K383, F385, |
| T329 | D386, V392, I396 I362, D363, |
| | G364, W365, Q382, |
| H45, V47, N48, | T385, Q386, I389, T393, V396, |
| L49, N304, S305, | N397, I400; |
| S306, L307, P308, | |
| T333; | |
| Y114, T147, N149, | |
| G150, G151, S152, | |
| N153, S161, | |
| W169, T171, | |
| K172, Q205, E206, | |
| T208, S209, L210, | |
| L242 | |
| | FIEGGWTGMVDGWYGYHH |
| | (SEQ ID NO: 10), part |
| | of fusion peptide |
| E341; | E360, G361, I363, D364, R370, |
| | E375, T377, G378, Q379, A380, |
| | A381, L383, N491, E495 |
| P37, E341, K342 | E360, G361, I363, D364, R370, |
| | T377, Q379, L383 |
| Y115, T148, N150, | |
| G151, G152, S153, | |
| S154, S162, W170, | |
| Y172, K173, S174, | |
| G175, N210, L211, | |
| L243 | |
| Mutations that | M360A,D362A, G363A, |
| reduce binding: | W364A, T384A, V395A, N396A, |
| H25A, N336A, | E400A (sequence numbering |
| P338A | refers to A/PR/8/34 |
| H34, V36, N54, | T334, V363, D364, G365, |
| S70, K292, N294, | W366, Q379, A380, D382, |
| P305, D307, | L383, K384, T386, Q387, I390, |
| | D391, I393, N394, G395, L397, |
| | N398, I401, K403, T404, N405, |
| | R498 |
| K147, V149, S159, | |
| W167, K170, | |
| S173, H197, P200, | |
| T201, T203, D204, | |
| Q206, S207, L208, | |
| Q210, K236, D239, | |
| A241, G242 | |
| S138, S139, P141, | |
| N142, K171, G172, | |
| S173, S174, Y175, | |
| P176, K177, S179, | |
| K180, S181, V183, | |
| N211, E260, T262 | |
| K147, V149, T150, | |
| A151, G157, A158, | |
| K159, W167, | |
| A203, D204, Q206, | |
| S207, L208, K236, | |
| D239, Q240 | |
| G147, V148, S149, | |
| A150, W166, | |
| T168, G169, N171, | |
| G172, L173, N200, | |
| G202, D203, R205, | |
| A206, L207, K235, | |
| D238, R239, E240 | |
| G147, V148, S149, | |
| A150, W166, | |
| T168, N171, G172, | |
| L173, N200, G202, | |
| D203, R205, A206, | |
| L207, H209, K232, | |
| D238 | |
| T89, A90, S127, | |
| E129, R130, E132, | |
| P135, S138, K180, | |
| K186, K188, Y270, | |
| A273 | |
| E129, R130, F131, | |
| E132, P135, T137, | |
| S138, K180, S181, | |
| I183, Y270 | |
| T138, Q139, T141, | |
| G144, G145, S146, | |
| R147, P155, T165, | |
| K166, K167, G168, | |
| S169, T203, L204 | |
| T141, T143, G144, | |
| G145, S146, R147, | |
| A150, G153, N154, | |
| P155, W163, T165, | |
| E166, G168, T199, | |
| E200, T203, L204, | |
| Q236 | |
| T141, T143, G144, | |
| G145, S146, R147, | |
| P155, W163, T165, | |
| K166, G168, S169, | |
| N196, D197, E198, | |
| T199, E200, R202, | |
| T203, L204, K232, | |
| G235, L236, G237, | |
| S238 | |
| Within 189-197 | |
| NEDKLYIWG | |
| (SEQ ID NO: 11) | |
| and 227-239 | |
| SSRISIYWTIVKP | |
| (SEQ ID NO: 12) | |
| S107, K108, N187, | |
| D188, N189, P255, | |
| G256, S286, D287, | |
| A288, P289, P300, | |
| N301 | |
| 68, 112, 137, 143, | |
| 251, 254, and 255 | |
| 118, 121, 161, 164, | |
| and 167 (mature | |
| H5 numbering) | |
| P134, S137, K177, | |
| Y180, T183 (IEDB | |
| (SEQ ID NO: 13)) | |
| 271-281, centered | |
| on 272-275 | |
| I132, I133, P134, | |
| W138, S139, | |
| Y180, T183 | |
| D59, D61, V63, | |
| K64, R69, E85, | |
| N88, P90, E91, | |
| H126, E128, E286, | |
| Y287, N289, N291 | |
| 88-90 NVP and | |
| 126-131 HFEKIQ | |
| (SEQ ID NO: 14) | |
| T52, K53, S54, | |
| H55, F56, K67, | |
| N74, T76, D77, | |
| H100, E101, G300, | |
| S301, L302, P303 | |
| T52, K53, Y55, | |
| F56, K67, L73, | |
| N74, C75, T76, | |
| D77, H100, E101, | |
| V105, G299, S300, | |
| L301, P302, I304 | |

The publications in Table Z are incorporated by reference in their entireties.

The disclosure also relates to aptamers in compositions that are simultaneously administered with antigens that comprise immunotherapeutic epitopes. While the aptamers of the disclosed compositions are designed to bind or be specific for immunodominant epitopes, the aptamers should be designed such that they do not bind to or mask immunotherapeutic epitopes at under biologically relevant conditions that allow for exposure and/or binding. While the aptamer may have any of the K_{D} levels above associated with their binding to the immunodominant epitopes, in some embodiments, the aptamers also have K_{D} value that are high relative to the immunotherapeutic epitopes that is a therapeutic target of the disclosure. In some embodiments, the aptamer comprises a K_{D} value of no lower than about 500 nM relative to the immunotherapeutic epitope. In some embodiments, the aptamer comprises a K_{D} value of no lower than about 550 nM relative to the immunotherapeutic epitope. In some embodiments, the aptamer comprises a K_{D} value of no lower than about 600 nM relative to the immunotherapeutic epitope. In some embodiments, the aptamer comprises a K_{D} value of no lower than about 650 nM relative to the immunotherapeutic epitope. In some embodiments, the aptamer comprises a K_{D} value of no lower than about 700 nM relative to the immunotherapeutic epitope. In some embodiments, the aptamer comprises a K_{D} value of no lower than about 750 nM relative to the immunotherapeutic epitope. In some embodiments, the aptamer comprises a K_{D} value of no lower than about 800 nM relative to the immunotherapeutic epitope. In some embodiments, the aptamer comprises a K_{D} value of no lower than about 850 nM relative to the immunotherapeutic epitope. In some embodiments, the aptamer comprises a K_{D} value of no lower than about 900 nM relative to the immunotherapeutic epitope. In some embodiments, the aptamer comprises a K_{D} value of no lower than about 950 nM relative to the immunotherapeutic epitope. In some embodiments, the aptamer comprises a K_{D} value of no lower than about 1000 nM relative to the immunotherapeutic epitope. In some embodiments, the aptamer comprises a K_{D} value of no lower than about 1500 nM relative to the immunotherapeutic epitope.

In some embodiments, the composition comprises an aptamer that comprises at least about 70% sequence identity to one or a plurality of sequences chosen from Table X.

TABLE X
Non-Limiting Examples of Aptamer Sequences
SARS-COV2 epitopes
5′ TCGGGCGAGTCGTCTGN₅₀CCGCATCCTCCTCCC 3′
(SEQ ID NO: 15)
5′ TCGGGCGAGTCGTCTGagctgtcgagtcgaaggttgacgtagcg
ccgtagcatcgatgcccgtttgCCGCATCCTCCTCCC 3′
(SEQ ID NO: 16)
5′ TCGGGCGAGTCGTCTGagctgtcgagtcgaagcatgacgtagcg
ccgtagcatcgttgcccgtttgCCGCATCCTCCTCCC 3′
(SEQ ID NO: 17)
5′ TCGGGCGAGTCGTCTGagctgtcgagtttaaggttgacgtagcg
ccgtagcatcgcctcccgtttgCCGCATCCTCCTCCC 3′
(SEQ ID NO: 18)
HIV-1 epitopes
5′ TCGGGCGAGTCGTCTGN₅₀CCGCATCCTCCTCCC 3′
(SEQ ID NO: 15)
5′ TCGGGCGAGTCGTCTGcctagtcgagtcgaaggttgacgtagcg
ccgtaaggtcgatgcccgtttgCCGCATCCTCCTCCC 3′
(SEQ ID NO: 19)
5′ TCGGGCGAGTCGTCTGagctgtacagtcgaaggttggcgtagcg
cctaagcatcgatgagcgtttgCCGCATCCTCCTCCC 3′
(SEQ ID NO: 20)
Influenza epitopes
5′ TCGGGCGAGTCGTCTGN₅₀CCGCATCCTCCTCCC 3′
(SEQ ID NO: 15)
5′ TCGGGCGAGTCGTCTGagctgacaagtcgaaggttgacgtttcg
ccgtagcatcgacgcccgtttgCCGCATCCTCCTCCC 3′
(SEQ ID NO: 21)
5′ TCGGGCGAGTCGTCTGagctgacgagtcgaaggttgacgtttcg
ccgtagcatcgatgccgatttgCCGCATCCTCCTCCC 3′
(SEQ ID NO: 22)

The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:15, wherein N₅₀ is a string of any 50 nucleotides specific to the epitope portion of the immunodominant epitope targeted for masking. The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:21, wherein N50 is a string of nucleotides specific to the epitope portion of the immunodominant epitope targeted for masking an influenza epitope. The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO: 22, wherein N₅₀ is a string of nucleotides specific to the epitope portion of the immunodominant epitope targeted for masking an influenza epitope. The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:20, wherein N50 is a string of any 50 nucleotides specific to the epitope portion of the immunodominant epitope targeted for masking an HIV epitope. The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:19, wherein N50 is 50 nucleotides specific to the epitope portion of the immunodominant epitope targeted for masking an HIV epitope. The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:18, wherein N50 is 50 nucleotides specific to the epitope portion of the immunodominant epitope targeted for masking an COVID epitope. The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:17, wherein N50 is 50 nucleotides specific to the epitope portion of the immunodominant epitope targeted for masking a COVID epitope. The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:16, wherein N50 is 50 nucleotides specific to the epitope portion of the immunodominant epitope targeted for masking an COVID epitope.

The disclosure relates to pharmaceutical compositions comprising an aptamer disclosed herein; and a pharmaceutically acceptable carrier. Pharmaceutical “carrier” or “excipient”, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore, Md., 2006) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, the pharmaceutically acceptable excipient or carrier is at least about 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydro xymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

In some embodiments, the pharmaceutical compositions comprise a therapeutically effective amount of an aptamer; a vaccine or antigen and a pharmaceutically acceptable carrier or excipient. The antigens of the compositions may be designed to comprise an endogenous human protein, an endogenous viral protein or an antigen comprising a tumor-associated antigen. The protein, polypeptides or fragments thereof of the antigen of the vaccine may be encoded by a polynucleotide. In some embodiments the polynucleotide is an mRNA. In some embodiments the vaccine mRNA are chemically modified.

The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:23. The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:24.

The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:25.

The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:26.

The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:27.

The disclosure relates to compositions comprising aptamers comprising at least 70%, 75%, 80%, 85%, 90%, 91%, 92%, 93%, 94%, 95%, 96%, 97%, 98% or 99% sequence identity to SEQ ID NO:28.

### Methods

The disclosure relates to a method of enhancing an immune response against an immunotherapeutic epitope comprising exposing a cell to a composition comprising an aptamer specific for an immunodominant epitope of an antigen, wherein the antigen comprises the immunodominant epitope and an immunotherapeutic epitope. In some embodiments, the exposure steps may be performed simultaneously, in sequence or partially simultaneously or partially in sequence. In some embodiments, the antigen is exposed to the cell only after exposing the cell to an expressible nucleic acid sequence encoding the antigen comprising an epitope. In some embodiments, the immunodominant epitope and the immunotherapeutic epitopes are present on the same antigen polypeptide sequence, or present on a plurality of antigens or polypeptides. In some embodiments, the epitopes are encoded on one or a plurality of expressible nucleic acid sequences that encode separate or fused polypeptide sequence comprising the epitopes.

The disclosure relates to a method of enhancing an immune response against an immunotherapeutic epitope comprising exposing a cell to a composition comprising an aptamer specific for an immunodominant epitope and an antigen comprising the immunodominant epitope for a time period sufficient to inhibit the immune response elicited by the immunodominant epitope.

In some embodiments, the cell is a lymphocyte or an antigen presenting cell. In some embodiments, the cell is a B-cell, T-cell, astrocyte, macrophage, any of the foregoing derived from a stem cell. The methods may be performed in vivo within a subject or in vitro within a cell culture or cell culture system.

The disclosure is also related to a method of generating a B cell that expresses an antibody or antibody fragment capable of binding one or a plurality of immunotherapeutic epitopes, the method comprising: exposing a cell to a composition comprising an aptamer specific for a immunodominant epitope and an antigen comprising the immunodominant epitope; and a step of exposing the cell to an antigen comprising an immunotherapeutic epitope. In some embodiments, the immune response to the immunotherapeutic epitope is enhanced while the aptamer blocks or inhibits the cell from mounting a measurable immune response against the immunodominant epitope.

The disclosure also relates to a method of treating or preventing a disorder in a subject comprising administering to the subject one or a plurality of pharmaceutical compositions comprising a therapeutically effective amount of one or a plurality of aptamers disclosed herein; and one or a plurality of pharmaceutically acceptable carriers. In some embodiments, the pharmaceutical composition or compositions further comprise an antigen comprising an immunodominant epitope and/or an immunotherapeutic epitope, or nucleic acids encoding the same. The disclosed aptamers may target an immunodominant epitope that is known to be involved in the development of cancer or viral disease, for example, those epitopes disclosed in Table Y. In some embodiments, administration of the pharmaceutical composition comprising the aptamer to the subject results in reduction enhancement of a therapeutically effective antigen-specific immune response against an immunotherapeutic epitope in the subject. In some embodiments, administration of the pharmaceutical composition to the subject results in a reduction in serum levels of viral load or viral enzymes present in the serum after an infection. In some embodiments, administration of the pharmaceutical composition to the subject results in a decrease of viral load in the subject or prevention of infection of the virus in the subject. In some embodiments, the disorder is cancer or a viral infection.

Suitable methods of administering the pharmaceutical composition to the subject may include oral administration, parenteral delivery, including intramuscular, subcutaneous, intramedullary injections, as well as intrathecal, direct intraventricular, intravenous, or

intraperitoneal injections. In a particular embodiment, the pharmaceutical composition is administered by injection. In some embodiments, the pharmaceutical composition is administered by intravenous infusion. In some embodiments, the pharmaceutical composition is administered by electroporation.

Intracellular delivery of the one or plurality of disclosed aptamers and/or one or plurality of antigen comprising disclosed epitopes has the potential to induce immune responses for many therapeutic applications. Although lipid nanoparticles have shown considerable promise for the delivery of small interfering RNAs (siRNA), their utility as agents for mRNA delivery of sequence encoding antigens has only recently been investigated. The most common siRNA formulations contain four components: an amine-containing lipid or lipid-like material, phospholipid, cholesterol, and lipid-anchored polyethylene glycol, the relative ratios of which can have profound effects on the formulation potency. In some embodiments, the methods comprise a generalized strategy to optimize lipid nanoparticle formulations for mRNA delivery to the liver in vivo using Design of Experiment (DOE) methodologies including Definitive Screening and Fractional Factorial Designs. By simultaneously varying lipid ratios and structures, we developed an optimized formulation which increased the potency of erythropoietin-mRNA-loaded C12-200 lipid nanoparticles 7-fold relative to formulations previously used for siRNA delivery. Key features of this optimized formulation were the incorporation of 1,2-dioleoyl-sn-glycero-3-phosphoethanolamine (DOPE) and increased ionizable lipid: mRNA weight ratios. Interestingly, the optimized lipid nanoparticle formulation did not improve siRNA delivery, indicating differences in optimized formulation parameter design spaces for siRNA and mRNA.

The disclosure also relates to a method of identifying one or a plurality of immunotherapeutic epitopes on an antigen and a method of identifying one or more sequences of an antibody or antibody fragment capable of binding an immunotherapeutic epitope on an antigen comprising:
(a) exposing one or a plurality of cells to a composition comprising an aptamer specific for an immunodominant epitope and an antigen comprising the immunodominant epitope for a time period sufficient to inhibit the immune response elicited by the immunodominant epitope.
(b) exposing one or a plurality of cells to a composition comprising an antigen comprising an immunotherapeutic epitope for a time period sufficient to elicit an antigen-specific immune response against the immunotherapeutic epitope in the cell;
(c) isolating the one or plurality of cells; and
(d) isolating an antibody or antibody fragment specific for the immunotherapeutic epitope or a nucleic acid sequence encoding the antibody or antibody fragment.

The disclosure also relates to a method of identifying one or a plurality of immunotherapeutic epitopes on an antigen and a method of identifying one or more sequences of an antibody or antibody fragment capable of binding an immunotherapeutic epitope on an antigen comprising:
(a) exposing one or a plurality of cells to a composition comprising an aptamer specific for an immunodominant epitope of an antigen for a time period sufficient to inhibit the immune response elicited by the immunodominant epitope.
(b) exposing one or a plurality of cells to a composition comprising the antigen comprising an immunotherapeutic epitope for a time period sufficient to elicit an antigen-specific immune response against the immunotherapeutic epitope in the cell;
(c) isolating the one or plurality of cells; and
(d) isolating an antibody or antibody fragment specific for the immunotherapeutic epitope or a nucleic acid sequence encoding the antibody or antibody fragment. In some embodiments, the method further comprises sequencing the amino acid sequence of the antibody or antibody fragment in order to identify the CDR or amino acid sequence responsible for binding to a therapeutic epitope. In some embodiments, the method further comprises compiling the sequence of the CDR and/or the therapeutic epitope sequences in order to map or clone the therapeutic epitope sequences. Any of the methods disclosed herein can comprise any composition disclosed herein with any aptamer comprising at least about 70% sequence identity to SEQ ID NO: 15, 16, 17, 18, 19, 20, 21, 22, 23, 24, 25, 26, 27 or 28 or a functional fragment of SEQ ID NO: 15, 16, 17, 18, 19, 20, 21, 22, 23, 24, 25, 26, 27 or 28 comprising 75%, 80%, 85%, 90%, 91%, 92%, 93%, 94%, 95%, 96%, 97%, 98% or 99% sequence identity to SEQ ID NOs: 15, 16, 17, 18, 19, 20, 21, 22, 23, 24, 25, 26, 27 or 28.

In some embodiments, the cell is a lymphocyte or an antigen presenting cell. In some embodiments, the cell is a B-cell, T-cell, astrocyte, macrophage, any of the foregoing derived from a stem cell. The methods may be performed in vivo within a subject or in vitro within a cell culture or cell culture system. In some embodiments, the methods further comprise a step of sequencing the antibody or antibody fragment specific for the immunotherapeutic epitope or a the nucleic acid sequence encoding the antibody or antibody fragment. In this way, using this method, one can screen for a panel of antibody or antibody fragment that may be the most therapeutically effective by skewing the immunogenicity of an immunotherapeutic epitope as compared to or relative to the immunogenicity of an immunotherapeutic epitope after exposure of the antigens to a cell in the absence of the one or plurality of aptamers.

In some embodiments, the methods use a composition disclosed herein to mask an immunodominant epitope identified in TABLE Y. The methods relate to methods of enhancing an antigen-specific immune response again a viral antigen other than the immunodominant epitopes identified in TABLE Y by administering to a subject in need thereof a composition or pharmaceutical composition comprising a therapeutically effective amount of an aptamer disclosed herein in order to mask or inhibit eliciting an antigen-specific immune response against the immunodominant epitopes.

### EXAMPLES

Current vaccines for influenza, or the recently developed vaccines against SARS-CoV2, mostly rely on the induction of antibodies against non-conserved epitopes of a few viral variants; thus, the elicited immunity is not expected to be broadly protective. Remarkably, some HIV-1 infected individuals develop bNAbs against HIV-1 after 1-2 years of infection (6-8). Human bNAbs bind to conserved regions of the HIV-1 Envelope protein (Env), and confer protection against viral challenge when used in passive immunization experiments in macaques (9-11), suggesting that a vaccine that elicits this type of antibodies would be protective. Despite the observation of the existence of bNAbs, these approaches show some limitations. They require the identification and characterization of the epitopes to be silenced as they involve targeted protein modifications or mutations, or the use of monoclonal antibodies. In addition, protein modifications may have co-lateral effects on the conserved epitopes of interest, through changes in the conformation of the protein or the elimination of effective T-cell epitopes. PEGylation had only a modest effect to re-direct antibody responses in vivo, and the addition of extra glycans to the HIV-1 Env protein negatively affected protein stability and conformation. In summary, no reported strategy has been successful at focusing antibody responses to conserved epitopes of viral antigens, especially upon repeated immunization or sequential immunization.

### Results

To evaluate the suitability of aptamers for epitope masking, we have taken advantage of previously reported aptamers against HEL, SAV and His-tag.

HEL_Aptamer 1
(SEQ ID NO: 23)
TTTTTTATCAGGGCTAAAGAGTGCAGAGTTACTTAG
HEL_Aptamer 2
(SEQ ID NO: 24)
AGCAGCACAGAGGTCAGATGGCAGGTAAGCAGGCGGCTCACAAAACCAT
TCGCATGCGGC
HEL_Aptamer 3
(SEQ ID NO: 25)
GGGAATGGATCCACATCTACGAATTCATCAGGGCTAAAGAGTGCAGAGT
TACTTAGTTCACTGCAGACTT GACGAAGCTT
HEL_Aptamer 4
(SEQ ID NO: 26)
AGCAGCACAGAGGTCAGATGGCAGGTAAGCAGGCGGCTCACAAAACCAT
TCGCATGCGGCCCTATGCGT GCTACCGTGAA
SAV Aptamer
(SEQ ID NO: 27)
ATACCAGCTTATTCAATTCTATACTCCACTTTGCTATTTCTCGGTTCCT
TCACGCGCCGATCGCAGGCTGAT GAATTGAGATAGTAAGTGCAATCT
His tag Aptamer
(SEQ ID NO: 28)
GCTATGGGTGGTCTGGTTGGGATTGGCCCCGGGAGCTGGC

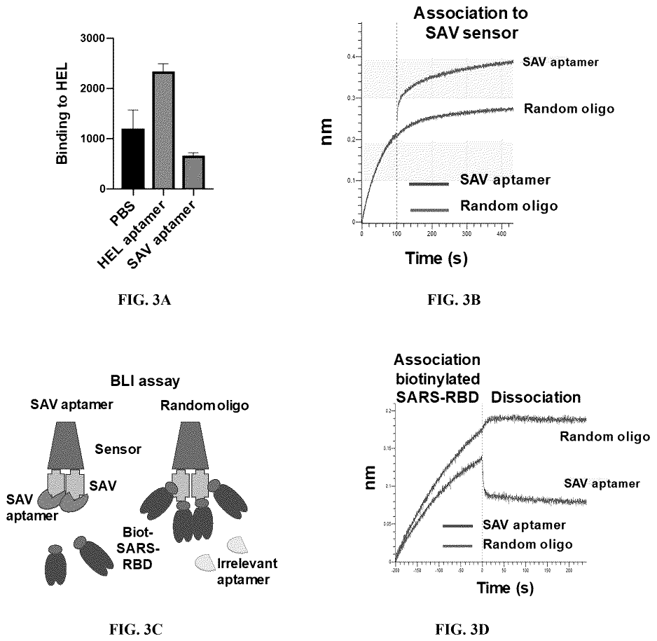

We have confirmed aptamer binding to HEL, SAV and His-tag through different assays including ELISAs, BLI and flow cytometry (FIGS. 3A-B and 4C-D). We have developed a BLI assay to prove that the binding of the SAV aptamer to SAV interferes with the binding of a biotinylated SARS-COV2-RBD antigen (FIGS. 3C and 3D). These results indicate that aptamers can mask epitopes in vitro.

To evaluate aptamers for epitope masking in vivo, we are doing preliminary experiments using HEL-aptamer, SAV-aptamer and Env-His-tag-aptamer complexes to immunize transgenic mice carrying an anti-HEL antibody (MD4 mice) and/or wild type mice. The analysis of the serologic responses elicited in the immunized mice, will inform about the capacity of aptamers to mask immunodominant epitopes of these proteins in vivo.

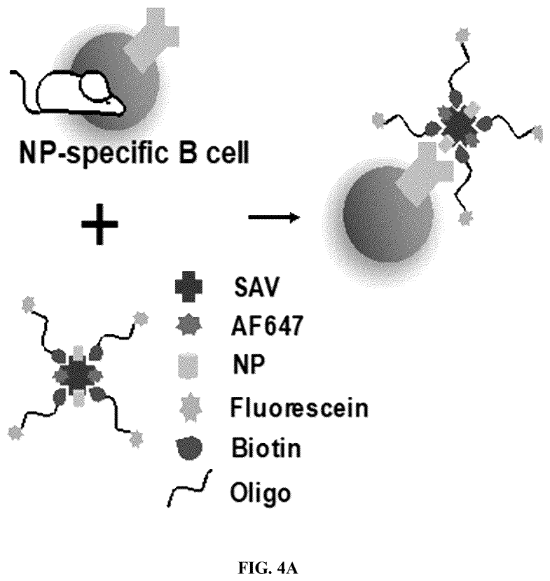

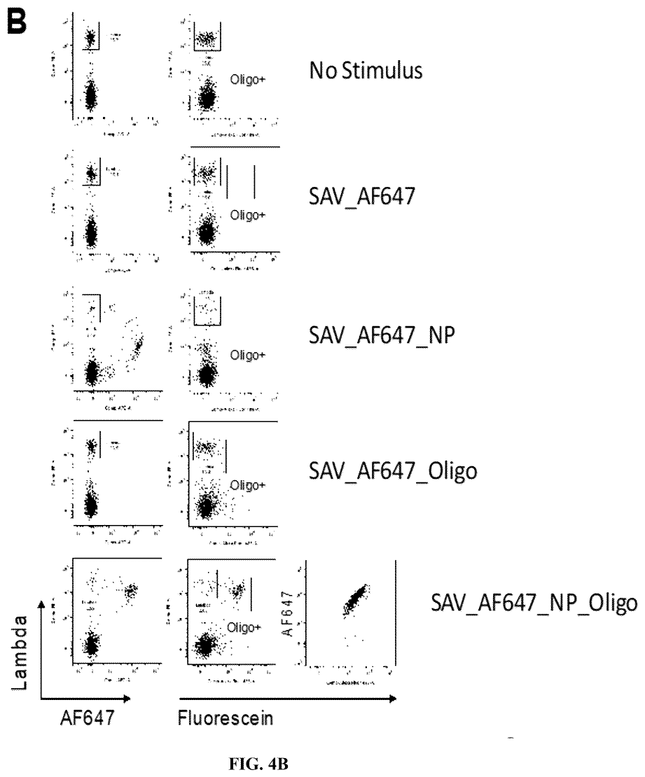

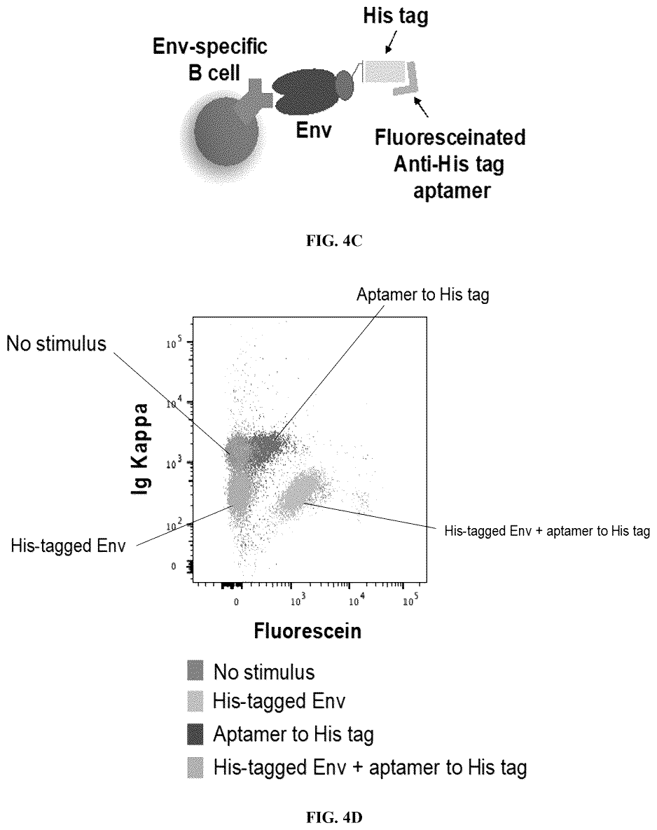

To investigate the suitability of aptamers to identify antigen-specific B cells, we have used a random 100-mer ssDNA oligo which is biotinylated at the 5′ end and fluoresceinated at the 3′ end. We have incubated this oligo with SAV conjugated to the 4-hydroxy-3-nitrophenyl acetyl (NP) hapten, and to the fluorophore Alexa Fluor 647 (SAV-AF647-NP) (FIG. 4A). We have used SAV-AF647-NP-oligo, and different variants lacking the NP or oligo components as controls, to stimulate transgenic B cells carrying an anti-NP specific antibody (B1-8 hi Lambda antibody) in vitro. Using this system, B cells acquiring the oligo through NP-BCR interaction and internalization, would be labelled with the AF647 and fluorescein fluorophores. In addition, lower surface expression of the lambda BCR would further indicate antigen-BCR interaction and internalization. In fact, the analysis of the stimulated B cells by flow cytometry showed that B cells stimulated with the SAV-AF647-NP-oligo, but not those stimulated with SAV-AF647, SAV-AF647-NP or SAV-AF647-oligo, internalized the fluoresceinated oligo, indicating that the internalization was dependent on specific NP-BCR interaction (FIG. 4B).

To investigate whether aptamers are internalized in complex with their specific antigens, and whether the aptamers can be detected inside the cells, we have prepared complexes of a His-tagged HIV-1 Env protein and a fluoresceinated anti-His-tag aptamer (FIG. 4C). We have used these complexes to stimulate B cells expressing an anti-Env antibody in vitro. Flow cytometry analysis has shown that only the B cells incubated with the Env-aptamer complexes, but not those incubated with the individual components, internalize the aptamer as evidenced by fluorescein detection and lower surface expression of BCR (FIG. 4D)

We propose to create tailored DNA-based covers to mask as much surface of the immunogen as possible, while the conserved epitope of interest remains exposed.

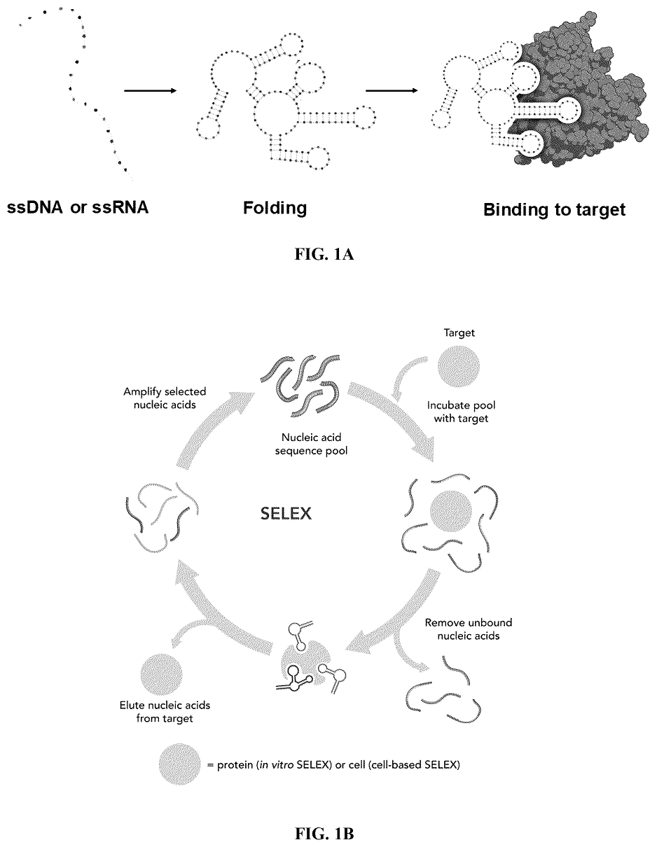

To do this, we will use aptamers, single stranded DNA (ssDNA) or RNA (ssRNA) molecules, which fold and adopt tertiary structures that can specifically recognize antigenic surfaces with high affinities comparable to those observed for antibodies (FIG. 1A). Aptamers have been used for many applications including clinical diagnostics, as therapeutic agents or as biosensors. In particular, aptamers have been exploited as inhibitors and activators, as carriers of therapeutic agents to target cells, or for recognition of cancers, pathogens or toxins among others. Aptamers present multiple advantages over antibodies; they can be produced in significantly shorter times and at a lower cost; they do not show batch-to-batch variability; they are highly modifiable, thermally stable and resistant to proteases; they have a higher target potential, from ions to live animals; and most importantly, they are non-immunogenic. We will use aptamers to cover potentially immunodominant epitopes of antigens aiming to redirect antibody responses to conserved epitopes of interest. Aptamer binding to antigen may also contribute to antigen stability when the antigen is a multimeric protein such as the HIV-1 Env or SARS-COV2 trimeric spike proteins. In this case, aptamers binding to interprotomer epitopes could maintain the trimers in a close conformation, further preventing the exposure of non-conserved immunodominant epitopes.

We will produce antigen-specific aptamers using the method called Systematic Evolution of Ligands by Exponential Enrichment (SELEX) (FIG. 1B). For SELEX, the antigen of interest is incubated with a pool of 1014-1016 random single-stranded oligonucleotides of typically 40-100 nucleotides containing a random region in the middle and fixed primer-annealing sequences on both ends. Non-binding oligonucleotides are discarded, and the antigen-binding oligonucleotides are eluted and amplified by PCR. This cycle is repeated multiple times, and after several rounds of selection, the resulting DNA sequences, with high affinity and specificity for the antigen, are enriched in the pool and sequenced. Either naked antigens, or antigens bound to Fabs protecting the conserved epitopes of interest, will be used as targets for SELEX. Selected aptamers will be characterized for their binding epitopes, and the antigen-aptamer complexes will be evaluated as immunogens in mice.

Aptamers to Identify Antigen Specific B cells and for Epitope Mapping

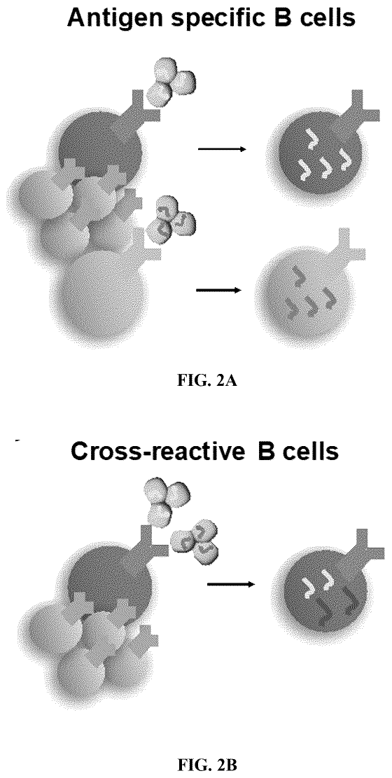

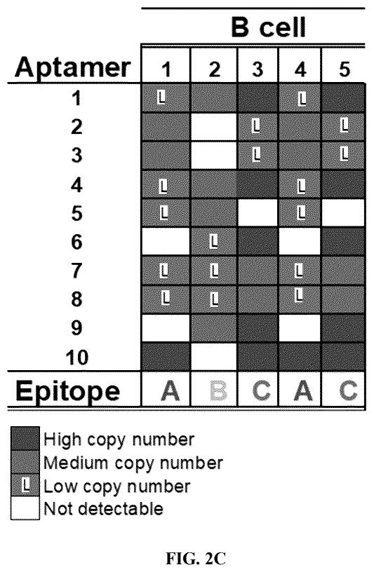

An additional bonus of the aptamer technology is that it can be used for high-throughput mapping of BCRs in a similar manner to the previously reported LIBRA-seq method. LIBRA-seq uses random ssDNA tails to barcode antigens, which are subsequently identified by Next Generation Sequencing (NGS) and used to identify antigen-specific B cells. Using our technology, we can take advantage of the aptamer specific binding to antigen. Aptamers will be internalized by the cell with their specific antigens upon antigen-BCR interaction, and the aptamer and BCR sequences will be then determined by NGS. The aptamer technology will allow to identify antigen- and epitope-specific B cells in large pools of cells, such as blood samples collected from virus infected individuals. This will be a great asset to studies aiming to isolate bNAbs. Incubating B cells with different antigen-aptamer complexes, followed by aptamer detection and sequencing in single B cells, will allow to classify B cells according to their antigen or epitope specificity (FIG. 2). The aptamer technology will be used as a screening method to identify antibodies of therapeutic and academic interest.

In summary, the aptamer technology in combination with single-B-cell antibody sequencing, will be very valuable for the characterization of antibody responses to vaccination and infection.

### Tailored DNA Suits for Immunogens

We will investigate the use of aptamers for two novel applications: 1) to silence immunodominant epitopes, and 2) to identify antigen-specific B cells.

We will initiate our studies using previously reported aptamers against proteins such as Hen Egg Lysozyme (HEL), streptavidin (SAV) and His tag to investigate the suitability of aptamers for the proposed applications. These aptamers were previously used for diagnostic purposes or to purify His-tagged recombinant proteins.

Epitope masking: We will perform ELISAs, Biolayer Interferometry (BLI) and other binding assays to confirm the binding of the aptamers to their corresponding targets, and to investigate whether aptamer binding interferes with epitope recognition. We will then evaluate the potential of the aptamers to mask immunodominant epitopes in vivo. To do this, we will immunize mice with the antigens or antigen-aptamer complexes and will interrogate whether aptamers prevent the elicitation of antibody responses. Once the function of aptamers to mask immunodominant epitopes is confirmed using existing tools and models, we will aim to produce and characterize aptamers against the HIV-1 Env protein. We will use SELEX to identify aptamer candidates for Env. We will determine their binding epitopes, and capability to limit the antibody responses to non-conserved epitopes when used as Env-aptamer immunogens in mice and eventually in macaques. We will produce aptamers for different available engineered and native-looking Env proteins such as RC1 and wild type BG505 respectively that we will use in sequential immunization protocols aiming to elicit anti HIV-1 bNAbs. We will produce aptamers and design combination of aptamers aiming to immunofocus the antibody response to conserved epitopes of Env including the V3-glycan epitope, the V1V2 epitope or the CD4bs epitope. We will immunize wild type mice, transgenic mice or macaques with Env-aptamer complexes. We will analyze the antibody responses elicited upon immunization with Env-aptamer complexes, and evaluate the effect of aptamer-mediated masking on the magnitude, specificity and quality of the response, i.e. on bNAb development. We will use ELISAs to characterize the binding of the elicited antibodies to the Env immunogens. We will use a combination of direct and competition ELISAs using the serum of the immunized animals to map the specific epitopes of the elicited antibody responses. For these ELISAs, we will use Env proteins carrying mutations in different epitopes to determine what mutations abrogate serum binding. For example, we will use Env proteins with mutations at positions 133, 137, 156, 160, 276, 301, 332, 368, 611, and the GDIR motif among others. Env mutations leading to reduced binding of the serum in ELISA will indicate that antibodies require intact residues at those positions for binding and will suggest that antibodies target an epitope containing those residues. We will use FACS to isolate single B cells from the spleen and lymph nodes collected from immunized animals, clone and produce their specific antibodies. We will produce the monoclonal antibodies expressed by these isolated B cells and evaluate their epitope specificity using ELISAs as explained above and Cryo-EM.

The results of our experiments will identify combinations of aptamers that in complex with their Env targets prevent antibody responses to non-conserved epitopes of Env upon immunization. Therefore, we expect an increase in antibody responses to conserved epitopes of Env such as the V3-glycan epitope, the V1V2 epitope or the CD4bs.

Identification of antigen-specific B cells: We will perform initial experiments to investigate whether aptamers are internalized together with antigens in a BCR-specific manner, and whether they can be detected upon internalization. We will use fluorescently labelled ssDNA oligos and antigen-aptamer complexes in combination with flow cytometry, PCR and NGS sequencing approaches to detect aptamers inside single B cells. Once available, we will use the newly designed HIV-1 Env aptamers, specific for different Env proteins or covering different antigenic regions of Env, to identify antigen and epitope-specific B cells in B cell homogenates derived from vaccinated animals. For this, we will immunize mice or macaques with Env immunogens, purify B cells by FACS and incubate them with different mixtures of Env-aptamer complexes. Next Generation Sequencing will be used to sequence the internalized aptamers and the antibody genes, and B cells will be classified according to their antigen binding patterns. Every Env-aptamer combination will allow B cell binding to specific epitopes. Identifying what aptamers were internalized by every B cell will indicate what epitopes were available for binding and provide information about the specificity of single B cells.

1. A method of vaccinating a subject comprising administering to a subject a composition comprising one or more aptamers targeting one or a plurality of immunodominant epitopes of one or more antigens.

2. The method of claim 1, wherein the method further comprises administering: (i) one or more antigens or nucleic acid sequences encoding the one or more antigens comprising an immunodominant epitope.

3. The method of claim 1 further comprising administering to the subject: (i) a nucleic acid sequence encoding one or a plurality of adjuvants; or (ii) one or a plurality of adjuvants.

4. (canceled)

5. The method of claim 1, wherein the one or more antigens comprise a viral antigen.

6. (canceled)

7. The method of claim 1, wherein the immunodominant epitopes are fold 1 and fold 2 of gp120 or fold 1 or 2 of gp41 or a fragment thereof that comprises at least about 70% sequence identity to the fold 1 and fold 2 of gp120 or fold 1 or 2 of gp41.

8. (canceled)

9. (canceled)

10. (canceled)

11. A method of enhancing the immunogenicity of one or a plurality of epitopes of an antigen in a subject in need thereof comprising administering to the subject an aptamer capable of associating with one or a plurality of immunodominant epitopes of the antigen.

12. The method of claim 11, wherein the method further comprises administering: (i) one or more antigens or (ii) one or more nucleic acid sequences encoding the one or more antigens comprising an immunodominant epitope.

13. The method of claim 11 further comprising administering to the subject: (i) a nucleic acid sequence encoding one or a plurality of adjuvants; or (ii) one or a plurality of adjuvants.

14. (canceled)

15. The method of claim 11, wherein the antigen is a viral antigen.

16. A method of enhancing the immunogenicity of one or a plurality of vaccines against one or a plurality of immunotherapeutic epitopes of an antigen in a subject in need thereof comprising administering to the subject an aptamer capable of associating with one or a plurality of immunodominant epitopes of the antigen.

17. The method of claim 16, wherein the method further comprises administering one or more of:
(i) one or more antigens;
(ii) one or more nucleic acid sequences encoding the one or more antigens comprising an immunodominant epitope;
(iii) a nucleic acid sequence encoding one or a plurality of adjuvants; and
(iv) one or a plurality of adjuvants.

18. (canceled)

19. (canceled)

20. The method of claim 16, wherein the antigen is a viral antigen.

21. A method of inducing or enhancing an immune response against an immunotherapeutic epitope in a subject in need thereof comprising administering to the subject an aptamer capable of associating with one or a plurality of immunodominant epitopes of an antigen.

22. The method of claim 21, wherein the antigen is a viral antigen.

23. (canceled)

24. The method of claim 21, wherein the one or a plurality of immunodominant epitopes are fold 1 and fold 2 of gp 120 or fold 1 or 2 of gp41 or a fragment thereof that comprises at least about 70% sequence identity to the fold 1 and fold 2 of gp120 or fold 1 or 2 of gp41.

25. (canceled)

26. The method of claim 21, wherein the aptamer sequence comprises at least about 70% sequence identity to GCAGGTGCAGGTTCACACTGGCAAG or a functional fragment thereof.

27. The method of claim 26, wherein the epitope comprises at least about 90% sequence identity to an HIV-1 epitope, at least about 90% sequence identity to an CoV2-epitope, or at least about 90% sequence identity to an influenza epitope.

28. (canceled)

29. (canceled)

30. A method of neutralizing viral infection or reducing viral load in a subject in need thereof comprising administering to the subject an aptamer capable of associating with one or a plurality of immunodominant epitopes of an antigen.

31. (canceled)

32. (canceled)

33. (canceled)

34. (canceled)

35. (canceled)

36. (canceled)

37. (canceled)

38. (canceled)

39. A method of generating neutralizing antibodies against an immunotherapeutic epitope in a subject in need thereof comprising administering to the subject an aptamer capable of associating with one or a plurality of immunodominant epitopes of an antigen.

40. (canceled)

41. (canceled)

42. (canceled)

43. (canceled)

44. A method of treating a subject in need thereof comprising administering to the subject a therapeutically effective amount of an aptamer capable of associating with one or a plurality of immunodominant epitopes of an antigen.

45. The method of claim 44 further comprising a step of administering to the subject: (i) a polypeptide that comprises an immunodominant epitope; or (ii) a nucleic acid that encodes a polypeptide that comprises an immunodominant epitope.

46. (canceled)

47. The method of claim 44, wherein the epitope is a viral epitope.

48. (canceled)

49. The method of claim 44, wherein the aptamer comprise at least 70% sequence identity to one or more of the sequences of Table X.

50. A method of reducing the immunogenicity of immunodominant epitopes of an antigen in a subject in need thereof comprising administering to the subject a therapeutically effective aptamer capable of associating with one or a plurality of immunodominant epitopes of an antigen.

51. (canceled)

52. The method of claim 50 further comprising a step of administering: (i) a polypeptide that comprises an immunotherapeutic epitope; or (ii) a nucleic acid that encodes a polypeptide that comprises an immunotherapeutic epitope.

53. The method of claim 50, wherein the epitope is a viral epitope.

54. (canceled)

55. The method of claim 50, wherein the aptamer comprises at least 70% sequence identity to one or more of the sequences of Table X.

56. A pharmaceutical composition comprising: a therapeutically effective amount of an aptamer comprising at least 70% sequence identity to one or more of the sequences of Table X; and a pharmaceutically acceptable carrier.

57. (canceled)
